(19) United States
(12) Patent Application Publication
(10) Pub. No.: US 2016/0021879 A1
(43) Pub. Date: Jan. 28, 2016

(54) SYNTHETIC LIPOAMINO ACID GLUCOSAMINE DERIVATIVES FOR IMPROVEMENT OF PLANT GROWTH AND YIELD

(71) Applicant: E.I. DU PONT DE NEMOURS AND COMPANY, Wilmington, DE (US)

(72) Inventors: Zheng-Zheng Huang, Hockessin, DE (US); Subramaniam Sabesan, Wilmington, DE (US); Zuohong Yin, West Chester, PA (US)

(73) Assignee: E. I. Du Pont De Nemours and Company, Wilmington, DE (US)

(21) Appl. No.: 14/773,585

(22) PCT Filed: Mar. 12, 2014
(86) PCT No.: PCT/US2014/023856

§ 371 Date: Sep. 8, 2015

Related U.S. Application Data
(60) Provisional application No. 61/782,017, filed on Mar. 14, 2013.

(51) Int. Cl. A01N 43/16; C07H 15/04

(52) U.S. Cl. CPC A01N 43/16; C07H 15/04

(57) ABSTRACT

The invention provides compounds, formulations and methods for improving plant emergence, growth and yield. More specifically, the present invention relates to compositions comprising the synthetic lipoamino acid glucosamine compounds of Formula I. These compounds may be applied to plant propagating materials, including seeds and other regenerable plant parts, including cuttings, bulbs, rhizomes and tubers. They may also be applied to foliage, or soil either prior to or following planting of plant propagating materials. Such applications may be made alone or in combination with fungicides, insecticides, nematicides and other agricultural agents used to improve plant growth and crop yield. The compounds of Formula I can improve the agronomic performance of a variety of crops including barley, canola, corn, potato, soybean and wheat.

### FIELD OF THE INVENTION

The present invention relates to formulations and methods of use of synthetic glucosamine derivatives for improving plant growth and crop yield.

### BACKGROUND

Signaling molecules are produced by rhizobia to initiate early stage root nodulation in leguminous plants. The resulting symbiotic relationship between the bacteria and plant provides reduced nitrogen to the plant and enhances growth or yield. Certain rhizobial inoculants and/or extracted natural rhizobia produced compounds are used to increase the productivity of a variety of leguminous crops, including soybeans, peanuts, alfalfa, and dry beans. These compounds may also be used to increase growth and yield in in non-leguminous crops such as corn.

Rhizobial inoculants and naturally derived compounds are currently produced via fermentation. The use of rhizobial inoculants, however, is constrained by several factors, including variability in production and cell viability in commercial formulations. Likewise, individual extracted compounds may be difficult to isolate from mixtures or are not amenable to economical methods of synthesis. Thus, there remains a need for a cost-effective alternative to these extracted compounds, as well as the opportunity to produce novel and efficacious derivative compounds. The present invention addresses this need.

### SUMMARY OF THE INVENTION

The invention provides formulations and methods for improving plant growth and crop yield. More specifically, the present invention relates to compositions comprising the synthetic lipoamino acid derivatized glucosamine compounds of Formula I. These compounds may be applied to plant propagating materials, including seeds and other regenerable plant parts, including cuttings, bulbs, rhizomes and tubers. They may also be applied to foliage, or soil either prior to or following planting of plant propagating materials. Such applications may be made alone or in combination with fungicides, insecticides, nematicides and other agricultural agents used to improve plant growth and crop yield.

### DETAILED DESCRIPTION OF THE INVENTION

The invention provides formulations and methods for improving plant growth and crop yield by treating plant propagating materials, foliage or soil with biologically effective amounts of the compounds of Formula I herein below:

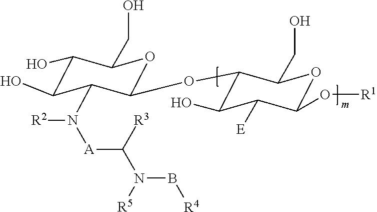

wherein m is 0, 1, 2, 3 or 4; A and B are selected from —C(O)—, —C(S)—, C(O)O—, —C(O)S—, —C(S)S—; E is selected from OH, NH₂, and NHC(O)CH₃; R¹ is selected from a linear or branched, saturated or unsaturated, hydrocarbon-based chain containing from 1 to 20 carbon atoms, arylene, or substituted arylene; R² and R⁵ is selected from H and C1-20 alkyl; R³ is selected from any side-chain of natural or unnatural amino acids, including a hydrogen, C1-6 alkyl, an aryl, and a halogen; and R⁴ is selected from a linear or branched, saturated or unsaturated, heteroatom substituted or non-heteroatom substituted hydrocarbon-based chain containing from 1 to 20 carbon atoms, arylene, or substituted arylene.

Specific structures based on formula 1 shown herein are illustrated based on m=0, whereas, in accordance with formula 1, one of skill in the art should also understand that m may equal any of 0, 1, 2, 3, or 4.

Certain embodiments of the present invention relates to the lipo glycine linked synthetic glucosamine derivatives shown below:

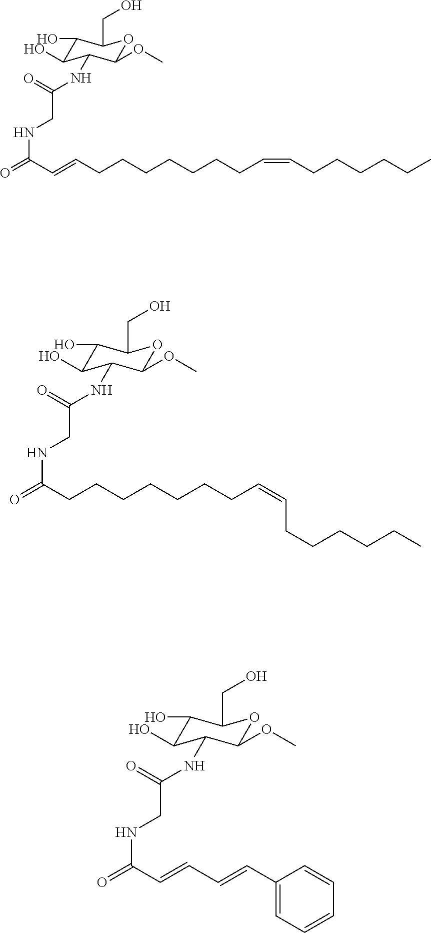 [SMILES: CCCCCC/C=C\CCCCCCC/C=C/C(=O)NCC(=O)NC1C(OC)OC(CO)C(O)C1O ; CCCCCC/C=C\CCCCCCCC(=O)NCC(=O)NC1C(OC)OC(CO)C(O)C1O ; COC1OC(CO)C(O)C(O)C1NC(=O)CNC(=O)/C=C/C=C/c1ccccc1]

In other embodiments, the present invention relates to compositions comprising the phenylalanine linked synthetic glucosamine derivative methyl-2-deoxy-2-({N-[(2E,4E)-5-phenylpenta-2,4-dienoyl]phenylalanyl}amino)hexopyranoside shown below:

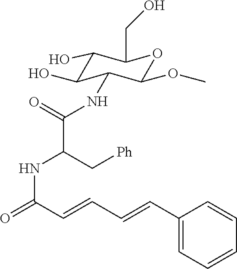 [SMILES: COC1OC(CO)C(O)C(O)C1NC(=O)C(Cc1ccccc1)NC(=O)/C=C/C=C/c1ccccc1]

In other embodiments the present invention relates to the aspartic acid linked synthetic glucosamine derivative shown below:

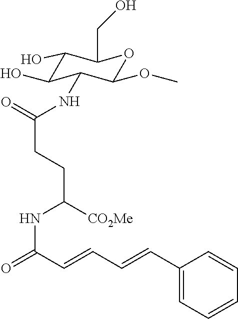 [SMILES: COC(=O)C(CCC(=O)NC1C(OC)OC(CO)C(O)C1O)NC(=O)/C=C/C=C/c1ccccc1]

In other embodiments the present invention relates to the glycine linked synthetic glucosamine derivatives shown below:

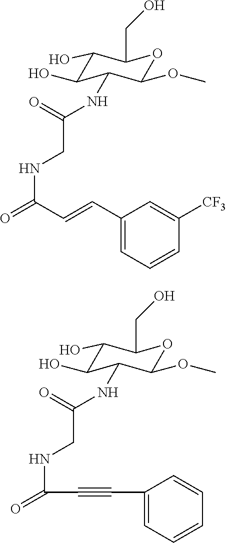

In another embodiment the present invention relates to the lipo glycine linked synthetic glucosamine derivative methyl 2-amino-2-deoxy-β-D-glucopyranosyl-(1→4)-2-actemido-2-deoxy-β-D-glucopyranosyl-(1→4)-2-acetamido-2-deoxy-β-D-glucopyranosyl-(1→4)-2acetamido-2-deoxy-β-D-glucopyranoside shown below:

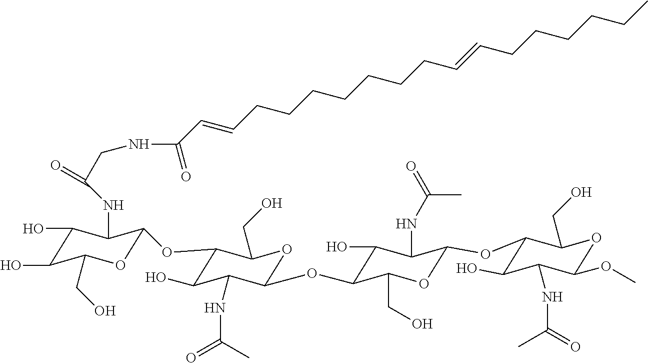 [SMILES: CCCCCC/C=C/CCCCCCC/C=C/C(=O)NCC(=O)NC1C(OC2C(CO)OC(OC3C(CO)OC(OC4C(CO)OC(OC)C(NC(C)=O)C4O)C(NC(C)=O)C3O)C(NC(C)=O)C2O)OC(CO)C(O)C1O]

The term “agricultural composition” as used herein comprises one or more substances formulated for at least one agricultural application. Agricultural applications are understood to include, but not be limited to, yield improvement, pest control, disease control and resistance to abiotic environmental stress.

As used herein the term “biologically effective amount” refers to that amount of a substance required to produce the desired effect on plant growth and yield. Effective amounts of the composition will depend on several factors, including treatment method, plant species, propagating material type and environmental conditions.

Foliage as defined in the present application includes all aerial plant organs, that is, the leaves, stems, flowers and fruit.

As used herein, “germination percentage” or “germination rate” refers the percentage of seeds that germinate after planting or being placed under conditions otherwise suitable for germination. The term “acceleration of germination” and its equivalents refer to an increase in the percent germination of experimentally treated seeds compared to seeds designated as experimental controls as a function of time, generally expressed as days after planting (DAP). In the Examples presented herein, seed germination rates were determined with laboratory-based germination assays conducted under optimum conditions for germination and conditions simulating salt and cold stress, wherein germination percentages were determined at specified DAP. General descriptions of seed germination tests can be found in the Handbook of Seed Technology for Genebanks, Volume I. Principles and Methodology, R. H. Ellis, T. D. Hong and E. H. Roberts, Eds., International Board for Plant Genetic resources, Rome, 1985, pp. 94-120 and the Seed Vigor Testing Handbook, Contribution No. 32 to the Handbook on Seed Testing prepared by the Seed Vigor Test Committee of the Association of Official Seed Analysts, 1983. Examples of seed cold and salt stress germination assays are respectively described in Burris and Navratil, Agronomy Journal, 71: 985-988 (1979) and Scialabba, et al., Seed Science & Technology, 27: 865-870 (1999).

Plant “growth” as used herein is defined by, but not limited to, measurements of seedling emergence, early growth, plant height, time to flowering, tillering (for grasses), days to maturity, vigor, biomass and yield.

As referred to in the present disclosure and claims, the term “propagating material” means a seed or regenerable plant part. The term “regenerable plant part” means a part of the plant other than a seed from which a whole plant may be grown or regenerated when the plant part is placed in agricultural or horticultural growing media such as moistened soil, peat moss, sand, vermiculite, perlite, rock wool, fiberglass, coconut husk fiber, tree fern fiber, and the like, or even a completely liquid medium such as water. Regenerable plant parts commonly include rhizomes, tubers, bulbs and corms of such geophytic plant species as potato, sweet potato, yam, onion, dahlia, tulip, narcissus, etc. Regenerable plant parts include plant parts that are divided (e.g., cut) to preserve their ability to grow into a new plant. Therefore regenerable plant parts include viable divisions of rhizomes, tubers, bulbs and corms which retain meristematic tissue, such as an eye. Regenerable plant parts can also include other plant parts such as cut or separated stems and leaves from which some species of plants can be grown using horticultural or agricultural growing media. As referred to in the present disclosure and claims, unless otherwise indicated, the term “seed” includes both unsprouted seeds and seeds in which the testa (seed coat) still surrounds part of the emerging shoot and root.

The term “rhizosphere” as defined herein refers to the area of soil that immediately surrounds and is affected by the plant's roots.

As used herein, the term “treating” means applying a biologically effective amount of a Formula I compound, or a composition containing a Formula I compound, to a seed or other plant propagating material, plant foliage or plant rhizosphere; related terms such as “treatment” are defined analogously.

As used herein, the terms “vigor” or “crop vigor” refer to the rate of growth, biomass volume, ground cover or foliage volume of a crop plant. In the Examples presented herein, “vigor” was determined by visual assessment and comparative scoring of plant growth parameters including height, width, and ground cover compared to control treatments.

The term “yield” as defined herein refers to the return of crop material per unit area obtained after harvesting a plant crop. An increase in crop yield refers to an increase in crop yield relative to an untreated control treatment. Crop materials include, but are not limited to, seeds, fruits, roots, tubers, leaves and types of crop biomass. Descriptions of field-plot techniques used to evaluate crop yield may be found in W. R. Fehr, Principles of Cultivar Development, McGraw-Hill, Inc., New York, N E, 1987, pp. 261-286 and references incorporated therein.

In one embodiment of the invention, the composition is applied as a seed treatment formulation. Such formulations typically contain from about 10⁻⁵M to 10⁻¹² M of the composition. In a preferred embodiment, formulations contain from about 10⁻⁶ M to 10⁻¹⁰ M of a Formula I compound. The locus of the propagating materials can be treated with a Formula I compound by many different methods. All that is needed is for a biologically effective amount of a Formula I compound to be applied on or sufficiently close to the propagating material so that it can be absorbed by the propagating material. The Formula I compound can be applied by such methods as drenching the growing medium including a propagating material with a solution or dispersion of a Formula I compound, mixing a Formula I compound with growing medium and planting a propagating material in the treated growing medium (e.g., nursery box treatments), or various forms of propagating material treatments whereby a Formula I compound is applied to a propagating material before it is planted in a growing medium.

In these methods a Formula I compound will generally be used as a formulation or composition with an agriculturally suitable carrier comprising at least one of a liquid diluent, a solid diluent or a surfactant. A wide variety of formulations are suitable for this invention, the most suitable types of formulations depend upon the method of application. As is well known to those skilled in the art, the purpose of formulation is to provide a safe and convenient means of transporting, measuring and dispensing the agricultural agent and also to optimize its efficacy.

Depending on the method of application useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and surfactant within the following approximate ranges that add up to 100 percent by weight.

| | Weight Percent | | |
|---|---|---|---|
| | Active | | |
| | Ingredient | Diluent | Surfactant |
| Water-Dispersible and | 5-90 | 0-94 | 1-15 |
| Water-soluble Granules, | | | |
| Tablets and Powders. | | | |
| Suspensions, Emulsions, | 5-50 | 40-95 | 0-15 |
| Solutions (including | | | |
| Emulsifiable Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Emulsifiers and Detergents and McCutcheon's Functional Materials (North America and International Editions, 2001), The Manufactuing Confection Publ.Co., Glen Rock, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, ethoxylated alcohols, ethoxylated alkylphenols, ethoxylated sorbitan fatty acid esters, ethoxylated amines, ethoxylated fatty acids, esters and oils, dialkyl sulfosuccinates, alkyl sulfates, alkylaryl sulfonates, organosilicones, N,N-dialkyltaurates, glycol esters, phosphate esters, lignin sulfonates, naphthalene sulfonate formaldehyde condensates, polycarboxylates, and block polymers including polyoxyethylene/polyoxypropylene block copolymers.

Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol, benzyl and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp. 147-48, Perry's Chemical Engineer's Handbook, 4th Ed., McGraw-Hill, New York, 1963, pp. 8-57 and following, and PCT Publication WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; and Hance et al., Weed Control Handbook, 8th Ed., Blackwell Scientific Publications, Oxford, 1989.

The compositions used for treating propagating materials, or plants grown therefrom, according to this invention can also comprise (besides the Formula I component) an effective amount of one or more other biologically active compounds or agents. Suitable additional compounds or agents include, but are not limited to, insecticides, fungicides, nematocides, bactericides, acaricides, entomopathogenic bacteria, viruses or fungi, growth regulators such as rooting stimulants, chemosterilants, repellents, attractants, pheromones, feeding stimulants and other signal compounds including apocarotenoids, flavonoids, jasmonates and strigolactones (Akiyama, et al., in Nature, 435:824-827 (2005); Harrison, in Ann. Rev. Microbiol., 59:19-42 (2005); Besserer, et al., in PLoS Biol., 4(7):e226 (2006); WO2009049747). These compounds can be formulated into a multi-component pesticide giving an even broader spectrum of agricultural utility than can be achieved with the Formula I component alone.

Examples of such biologically active compounds or agents with which compounds of this invention can be formulated are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including Bacillus thuringiensis (including ssp. aizawai and kurstaki), Bacillus thuringiensis delta-endotoxin, baculoviruses, and entomopathogenic bacteria, viruses and fungi. A general reference for these agricultural protectants is The Pesticide Manual, 12th Edition, C. D. S. Tomlin, Ed., British Crop Protection Council, Farnham, Surrey, U. K., 2000.

Preferred insecticides and acaricides for mixing with Formula I compounds include pyrethroids such as cypermethrin, cyhalothrin, cyfluthrin and beta-cyfluthrin, esfenvalerate, fenvalerate and tralomethrin; carbamates such as fenothicarb, methomyl, oxamyl and thiodicarb; neonicotinoids such as clothianidin, imidacloprid and thiacloprid; neuronal sodium channel blockers such as indoxacarb, insecticidal macrocyclic lactones such as spinosad, abamectin, avermectin and emamectin; γ-aminobutyric acid (GABA) antagonists such as endosulfan, ethiprole and fipronil; insecticidal ureas such as flufenoxuron and triflumuron; juvenile hormone mimics such as diofenolan and pyriproxyfen; pymetrozine; and amitraz. Preferred biological agents for mixing with compounds of this invention include Bacillus thuringiensis and Bacillus thuringiensis delta-endotoxin as well as naturally occurring and genetically modified viral insecticides including members of the family Baculoviridae as well as entomophagous fungi.

Preferred plant growth regulators for mixing with the Formula I compounds in compositions for treating stem cuttings are 1H-indole-3-acetic acid, 1H-indole-3-butanoic acid and 1-naphthaleneacetic acid and their agriculturally suitable salt, ester and amide derivatives, such as 1-napthaleneacetamide. Preferred fungicides for mixing with the Formula I compounds include fungicides useful as seed treatments such as thiram, maneb, mancozeb and captan.

For growing-medium drenches, the formulation needs to provide the Formula I compound, generally after dilution with water, in solution or as particles small enough to remain dispersed in the liquid. Water-dispersible or soluble powders, granules, tablets, emulsifiable concentrates, aqueous suspension concentrates and the like are formulations suitable for aqueous drenches of growing media. Drenches are most satisfactory for treating growing media that have relatively high porosity, such as light soils or artificial growing medium comprising porous materials such as peat moss, perlite, vermiculite and the like. The drench liquid comprising the Formula I compound can also be added to a liquid growing medium (i.e. hydroponics), which causes the Formula I compound to become part of the liquid growing medium. One skilled the art will appreciate that the amount of Formula I compound needed in the drench liquid for efficacy (i.e. biologically effective amount) will vary with several factors including, but not limited to, plant species, propagating material type and environmental conditions. The concentration of Formula I compound in the drench liquid is generally between about 10⁻⁵M to 10⁻¹²M of the composition, more typically between about 10⁻⁶ M to 10⁻¹⁰ M. One skilled in the art can easily determine the biologically effective concentration necessary for the desired level of efficacy.

For treating a growing medium a Formula I compound can also be applied by mixing it as a dry powder or granule formulation with the growing medium. Because this method of application does not require first dispersing or dissolving in water, the dry powder or granule formulations need not be highly dispersible or soluble. While in a nursery box the entire body of growing medium may be treated, in an agricultural field only the soil in the vicinity of the propagating material is typically treated for environmental and cost reasons. To minimize application effort and expense, a formulation of Formula I compound is most efficiently applied concurrently with propagating material planting (e.g., seeding). For in-furrow application, the Formula I formulation (most conveniently a granule formulation) is applied directly behind the planter shoe. For T-band application, the Formula I formulation is applied in a band over the row behind the planter shoe and behind or usually in front of the press wheel. One skilled the art will appreciate that the amount of Formula I compound needed in the growing medium locus for efficacy (i.e. biologically effective amount) will vary with several factors including, but not limited to, plant species, propagating material type and environmental conditions. The concentration of Formula I compound in the growing medium locus is generally between about 10⁻⁵M to 10⁻¹²M of the composition, more typically between about 10⁻⁶ M to 10⁻¹⁰ M. One skilled in the art can easily determine the biologically effective amount necessary for the desired level efficacy.

A propagating material can be directly treated by soaking it in a solution or dispersion of a Formula I compound. Although this application method is useful for propagating materials of all types, treatment of large seeds (e.g., having a mean diameter of at least 3 mm) is more effective than treatment of small seeds for providing efficacy. Treatment of propagating materials such as tubers, bulbs, corms, rhizomes and stem and leaf cuttings also can provide effective treatment of the developing plant in addition to the propagating material. The formulations useful for growing-medium drenches are generally also useful for soaking treatments. The soaking medium comprises a nonphytotoxic liquid, generally water-based although it may contain nonphytotoxic amounts of other solvents such as methanol, ethanol, isopropanol, ethylene glycol, propylene glycol, propylene carbonate, benzyl alcohol, dibasic esters, acetone, methyl acetate, ethyl acetate, cyclohexanone, dimethylsulfoxide and N-methylpyrrolidone, which may be useful for enhancing solubility of the Formula I compound and penetration into the propagating material. A surfactant can facilitate wetting of the propagating material and penetration of the Formula I compound. One skilled the art will appreciate that the amount of Formula I compound needed in the soaking medium for efficacy (i.e. biologically effective amount) will vary with several factors including, but not limited to, plant species, propagating material type and environmental conditions. The concentration of Formula I compound in the soaking liquid is generally between about 10⁻⁶ M to 10⁻¹² M of the composition, more typically between about 10⁻⁶ M to 10⁻¹⁰ M. One skilled in the art can easily determine the biologically effective concentration necessary for the desired level of efficacy. The soaking time can vary from one minute to one day or even longer. Indeed, the propagating material can remain in the treatment liquid while it is germinating or sprouting (e.g., sprouting of rice seeds prior to direct seeding). As shoot and root emerge through the testa (seed coat), the shoot and root directly contact the solution comprising the Formula I compound. For treatment of sprouting seeds of large-seeded crops such as rice, treatment times of about 8 to 48 hours, e.g., about 24 hours, is typical. Shorter times are most useful for treating small seeds.

A propagating material can also be coated with a composition comprising a biologically effective amount of a Formula I compound. The coatings of the invention are capable of effecting a slow release of a Formula I compound by diffusion into the propagating material and surrounding medium. Coatings include dry dusts or powders adhering to the propagating material by action of a sticking agent such as methylcellulose or gum arabic. Coatings can also be prepared from suspension concentrates, water-dispersible powders or emulsions that are suspended in water, sprayed on the propagating material in a tumbling device and then dried. Formula I compounds that are dissolved in the solvent can be sprayed on the tumbling propagating material and the solvent then evaporated. Such compositions preferably include ingredients promoting adhesion of the coating to the propagating material. The compositions may also contain surfactants promoting wetting of the propagating material. Solvents used must not be phytotoxic to the propagating material; generally water is used, but other volatile solvents with low phytotoxicity such as methanol, ethanol, methyl acetate, ethyl acetate, acetone, etc. may be employed alone or in combination. Volatile solvents are those with a normal boiling point less than about 100° C. Drying must be conducted in a way not to injure the propagating material or induce premature germination or sprouting.

The thickness of coatings can vary from adhering dusts to thin films to pellet layers about 0.5 to 5 mm thick. Propagating material coatings of this invention can comprise more than one adhering layer, only one of which need comprise a Formula I compound. Generally pellets are most satisfactory for small seeds, because their ability to provide a biologically effective amount of a Formula I compound is not limited by the surface area of the seed, and pelleting small seeds also facilitates seed transfer and planting operations. Because of their larger size and surface area, large seeds and bulbs, tubers, corms and rhizomes and their viable cuttings are generally not pelleted, but instead coated with powders or thin films.

Propagating materials contacted with compounds of Formula I in accordance to this invention include seeds. Suitable seeds include seeds of wheat, durum wheat, barley, oat, rye, maize, sorghum, rice, wild rice, cotton, flax, sunflower, soybean, garden bean, lima bean, broad bean, garden pea, peanut, alfalfa, beet, garden lettuce, rapeseed, cole crop, turnip, leaf mustard, black mustard, tomato, potato, pepper, eggplant, tobacco, cucumber, muskmelon, watermelon, squash, carrot, zinnia, cosmos, chrysanthemum, sweet scabious, snapdragon, gerbera, babys-breath, statice, blazing star, lisianthus, yarrow, marigold, pansy, impatiens, petunia, geranium and coleus. Of note are seeds of cotton, maize, soybean and rice. Propagating materials contacted with compounds of Formula I in accordance to this invention also include rhizomes, tubers, bulbs or corms, or viable divisions thereof. Suitable rhizomes, tubers, bulbs and corms, or viable divisions thereof include those of potato, sweet potato, yam, garden onion, tulip, gladiolus, lily, narcissus, dahlia, iris, crocus, anemone, hyacinth, grape-hyacinth, freesia, ornamental onion, wood-sorrel, squill, cyclamen, glory-of-the-snow, striped squill, calla lily, gloxinia and tuberous begonia. Of note are rhizomes, tubers, bulbs and corms, or viable division thereof of potato, sweet potato, garden onion, tulip, daffodil, crocus and hyacinth. Propagating materials contacted with compounds of Formula I in accordance to this invention also include stems and leaf cuttings.

One embodiment of a propagating material contacted with a Formula I compound is a propagating material coated with a composition comprising a compound of Formula I and a film former or adhesive agent. Compositions of this invention which comprise a biologically effective amount of a compound of Formula I and a film former or adhesive agent, can further comprise an effective amount of at least one additional biologically active compound or agent. Of note are compositions comprising (in addition to the Formula I component and the film former or adhesive agent) an arthropodicides of the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics. Also of note are compositions comprising (in addition to the Formula I component and the film former or adhesive agent) at least one additional biologically active compound or agent selected from the group consisting of abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad; and biological agents such as Bacillus thuringiensis (including ssp. aizawai and kurstaki), Bacillus thuringiensis delta-endotoxin, baculoviruses, and entomopathogenic bacteria, viruses and fungi. Also of note are compositions comprising (in addition to the Formula I component and the film former or adhesive agent) at least one additional biologically active compound or agent selected from fungicides of the group consisting of acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin (especially compositions wherein the at least one additional biologically active compound or agent is selected from fungicides in the group consisting of thiram, maneb, mancozeb and captan).

Generally a propagating material coating of the invention comprises a compound of Formula I, a film former or sticking agent. The coating may further comprise formulation aids such as a dispersant, a surfactant, a carrier and optionally an antifoam and dye. One skilled the art will appreciate that the amount of Formula I compound needed for efficacy (i.e. biologically effective amount) will vary with several factors including, but not limited to, plant species, propagating material type and environmental conditions. The coating needs to not inhibit germination or sprouting of the propagating material.

The film former or adhesive agent component of the propagating material coating is composed preferably of an adhesive polymer that may be natural or synthetic and is without phytotoxic effect on the propagating material to be coated. The film former or sticking agent may be selected from polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxymethylcelluloses, hydroxy-propylcellulose, hydroxymethylpropylcelluloses, polyvinylpyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. Preferred film formers and adhesive agents include polymers and copolymers of vinyl acetate, poly-vinylpyrrolidone-vinyl acetate copolymer and water-soluble waxes. Particularly preferred are polyvinylpyrrolidone-vinyl acetate copolymers and water-soluble waxes. The above-identified polymers include those known in the art and for example some are identified as Agrimer® VA 6 and Licowax® KST. The amount of film former or sticking agent in the formulation is generally in the range of about 0.001 to 100% of the weight of the propagating material. For large seeds the amount of film former or sticking agent is typically in the range of about 0.05 to 5% of the seed weight; for small seeds the amount is typically in the range of about 1 to 100%, but can be greater than 100% of seed weight in pelleting. For other propagating materials the amount of film former or sticking agent is typically in the range of 0.001 to 2% of the propagating material weight.

Materials known as formulation aids may also be used in propagating material treatment coatings of the invention and are well known to those skilled in the art. Formulation aids assist in the production or process of propagating material treatment and include, but are not limited, to dispersants, surfactants, carriers, antifoams and dyes. Useful dispersants can include highly water-soluble anionic surfactants like Borresperse™ CA, Morwet® D425 and the like. Useful surfactants can include highly water-soluble nonionic surfactants like Pluronic® F108, Brij® 78 and the like. Useful carriers can include liquids like water and oils which are water-soluble such as alcohols. Useful carriers can also include fillers like woodflours, clays, activated carbon, diatomaceous earth, fine-grain inorganic solids, calcium carbonate and the like. Clays and inorganic solids which may be used include calcium bentonite, kaolin, china clay, talc, perlite, mica, vermiculite, silicas, quartz powder, montmorillonite and mixtures thereof. Antifoams can include water dispersible liquids comprising polyorganic siloxanes like Rhodorsil® 416. Dyes can include water dispersible liquid colorant compositions like Pro-Ized® Colorant Red. One skilled in the art will appreciate that this is a non-exhaustive list of formulation aids and that other recognized materials may be used depending on the propagating material to be coated and the compound of Formula I used in the coating. Suitable examples of formulation aids include those listed herein and those listed in McCutcheon's 2001, Volume 2: Functional Materials, published by MC Publishing Company. The amount of formulation aids used may vary, but generally the weight of the components will be in the range of about 0.001 to 10000% of the propagating material weight, with the percentages above 100% being mainly used for pelleting small seed. For nonpelleted seed generally the amount of formulating aids is about 0.01 to 45% of the seed weight and typically about 0.1 to 15% of the seed weight. For propagating materials other than seeds, the amount of formulation aids generally is about 0.001 to 10% of the propagating material weight.

Conventional means of applying seed coatings may be used to carry out the coating of the invention. Dusts or powders may be applied by tumbling the propagating material with a formulation comprising a Formula I compound and a sticking agent to cause the dust or powder to adhere to the propagating material and not fall off during packaging or transportation. Dusts or powders can also be applied by adding the dust or powder directly to the tumbling bed of propagating materials, followed by spraying a carrier liquid onto the seed and drying. Dusts and powders comprising a Formula I compound can also be applied by treating (e.g., dipping) at least a portion of the propagating material with a solvent such as water, optionally comprising a sticking agent, and dipping the treated portion into a supply of the dry dust or powder. This method can be particularly useful for coating stem cuttings. Propagating materials can also be dipped into compositions comprising Formula I formulations of wetted powders, solutions, suspoemulsions, emulfiable concentrates and emulsions in water, and then dried or directly planted in the growing medium. Propagating materials such as bulbs, tubers, corms and rhizomes typically need only a single coating layer to provide a biologically effective amount of a Formula I compound.

Propagating materials may also be coated by spraying a suspension concentrate directly into a tumbling bed of propagating materials and then drying the propagating materials. Alternatively, other formulation types like wetted powders, solutions, suspoemulsions, emulsifiable concentrates and emulsions in water may be sprayed on the propagating materials. This process is particularly useful for applying film coatings to seeds. Various coating machines and processes are available to one skilled in the art. Suitable processes include those listed in P. Kosters et al., Seed Treatment: Progress and Prospects, 1994 BCPC Monograph No. 57 and the references listed therein. Three well-known techniques include the use of drum coaters, fluidized bed techniques and spouted beds. Propagating materials such as seeds may be presized prior to coating. After coating the propagating materials are dried and then optionally sized by transfer to a sizing machine. These machines are known in the art for example, as a typical machine used when sizing corn (maize) seed in the industry.

For coating seed, the seed and coating material are mixed in any variety of conventional seed coating apparatus. The rate of rolling and coating application depends upon the seed. For large oblong seeds such as those of cotton, a satisfactory seed coating apparatus comprises a rotating type pan with lifting vanes turned at sufficient rpm to maintain a rolling action of the seed, facilitating uniform coverage. For seed coating formulations applied as liquids, the seed coating must be applied over sufficient time to allow drying to minimize clumping of the seed. Using forced air or heated forced air can facilitate an increased rate of application. One skilled in the art will also recognize that this process may be a batch or continuous process. As the name implies, a continuous process allows the seeds to flow continuously throughout the product run. New seeds enter the pan in a steady stream to replace coated seeds exiting the pan.

The seed coating process of the present invention is not limited to thin film coating and may also include seed pelleting. The pelleting process typically increases the seed weight from 2 to 100 times and can be used to also improve the shape of the seed for use in mechanical seeders. Pelleting compositions generally contain a solid diluent, which is typically an insoluble particulate material, such as clay, ground limestone, powdered silica, etc., to provide bulk in addition to a binder such as an artificial polymer (e.g., polyvinyl alcohol, hydrolyzed polyvinyl acetates, polyvinyl methyl ether, polyvinyl methyl ether-maleic anhydride copolymer, and polyvinylpyrrolidinone) or natural polymer (e.g., alginates, karaya gum, jaguar gum, tragacanth gum, polysaccharide gum, mucilage). After sufficient layers have been built up, the coat is dried and the pellets graded. A method for producing pellets is described in Agrow, The Seed Treatment Market, Chapter 3, PJB Publications Ltd., 1994.

Seed varieties and seeds with specific transgenic traits may be tested to determine which seed treatment options and application rates may complement such varieties and transgenic traits in order to enhance yield. Further, the good root establishment and early emergence that results from the proper use of the compound of formula I seed treatment may result in more efficient nitrogen use, a better ability to withstand drought and an overall increase in yield potential of a variety or varieties containing a certain trait when combined with a seed treatment.

In another embodiment of the invention, the composition is applied as a foliar formulation. Such formulations will generally include at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake. Effective foliar formulations will typically contain from about 10⁻⁵M to 10⁻¹²M of the composition. In a preferred embodiment, formulations contain from about 10⁻⁶ M to 10⁻¹⁰ M of the compound of formula I.

In another embodiment of the invention, the composition is applied to soil either prior to or following planting of plant propagating materials. Compositions can be applied as a soil drench of a liquid formulation, a granular formulation to the soil, a nursery box treatment or a dip of transplants. Of note is a composition of the present invention in the form of a soil drench liquid formulation. Of further note is this method wherein the environment is soil and the composition is applied to the soil as a soil drench formulation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. Effective soil formulations will typically contain from about 10⁻⁵M to 10⁻¹²M of the composition. In a preferred embodiment, formulations contain from about 10⁻⁶ M to 10⁻¹⁰ M of the compound of formula I.

The method of this invention is applicable to virtually all plant species. Seeds that can be treated include, for example, wheat (Triticum aestivum L.), durum wheat (Triticum durum Desf.), barley (Hordeum vulgare L.), oat (Avena sativa L.), rye (Secale cereale L.), maize (Zea mays L.), sorghum (Sorghum vulgare Pers.), rice (Oryza sativa L.), wild rice (Zizania aquatica L.), millet (Eleusine coracana, Panicum miliaceum), cotton (Gossypium barbadense L. and G. hirsutum L.), flax (Linum usitatissimum L.), sunflower (Helianthus annuus L.), soybean (Glycine max Merr.), garden bean (Phaseolus vulgaris L.), lima bean (Phaseolus limensis Macf.), broad bean (Vicia faba L.), garden pea (Pisum sativum L.), peanut (Arachis hypogaea L.), alfalfa (Medicago sativa L.), beet (Beta vulgaris L.), garden lettuce (Lactuca sativa L.), rapeseed (Brassica rapa L. and B. napus L.), cole crops such as cabbage, cauliflower and broccoli (Brassica oleracea L.), turnip (Brassica rapa L.), leaf (oriental) mustard (Brassica juncea Coss.), black mustard (Brassica nigra Koch), tomato (Lycopersicon esculentum Mill.), potato (Solanum tuberosum L.), pepper (Capsicum frutescens L.), eggplant (Solanum melongena L.), tobacco (Nicotiana tabacum), cucumber (Cucumis sativus L.), muskmelon (Cucumis melo L.), watermelon (Citrullus vulgaris Schrad.), squash (Curcurbita pepo L., C. moschata Duchesne. and C. maxima Duchesne.), carrot (Daucus carota L.), zinnia (Zinnia elegans Jacq.), cosmos (e.g., Cosmos bipinnatus Cay.), chrysanthemum (Chrysanthemum spp.), sweet scabious (Scabiosa atropurpurea L.), snapdragon (Antirrhinum majus L.), gerbera (Gerbera jamesonii Bolus), babys-breath (Gypsophila paniculata L., G. repens L. and G. elegans Bieb.), statice (e.g., Limonium sinuatum Mill., L. sinense Kuntze.), blazing star (e.g., Liatris spicata Willd., L. pycnostachya Michx., L. scariosa Willd.), lisianthus (e.g., Eustoma grandiflorum (Raf.) Shinn), yarrow (e.g., Achillea filipendulina Lam., A. millefolium L.), marigold (e.g., Tagetes patula L., T. erecta L.), pansy (e.g., Viola cornuta L., V. tricolor L.), impatiens (e.g., Impatiens balsamina L.) petunia (Petunia spp.), geranium (Geranium spp.) and coleus (e.g., Solenostemon scutellarioides (L.) Codd). Not only seeds, but also rhizomes, tubers, bulbs or corms, including viable cuttings thereof, can be treated according to the invention from, for example, potato (Solanum tuberosum L.), sweet potato (Ipomoea batatas L.), yam (Dioscorea cayenensis Lam. and D. rotundata Poir.), garden onion (e.g., Allium cepa L.), tulip (Tulipa spp.), gladiolus (Gladiolus spp.), lily (Lilium spp.), narcissus (Narcissus spp.), dahlia (e.g., Dahlia pinnata Cay.), iris (Iris germanica L. and other species), crocus (Crocus spp.), anemone (Anemone spp.), hyacinth (Hyacinth spp.), grape-hyacinth (Muscari spp.), freesia (e.g., Freesia refracta Klatt., F. armstrongii W. Wats), ornamental onion (Allium spp.), wood-sorrel (Oxalis spp.), squill (Scilla peruviana L. and other species), cyclamen (Cyclamen persicum Mill. and other species), glory-of-the-snow (Chionodoxa luciliae Boiss. and other species), striped squill (Puschkinia scilloides Adams), calla lily (Zantedeschia aethiopica Spreng., Z. elliottiana Engler and other species), gloxinia (Sinnigia speciosa Benth. & Hook.) and tuberous begonia (Begonia tuberhybrida Voss.). Stem cuttings can be treated according to this invention include those from such plants as sugarcane (Saccharum officinarum L.), carnation (Dianthus caryophyllus L.), florists chrysanthemum (Chrysanthemum mortifolium Ramat.), begonia (Begonia spp.), geranium (Geranium spp.), coleus (e.g., Solenostemon scutellarioides (L.) Codd) and poinsettia (Euphorbia pulcherrima Willd.). Leaf cuttings which can be treated according to this invention include those from begonia (Begonia spp.), african-violet (e.g., Saintpaulia ionantha Wendl.) and sedum (Sedum spp.). The above recited cereal, vegetable, ornamental (including flower) and fruit crops are illustrative, and should not be considered limiting in any way. For reasons of economic importance, preferred embodiments of this invention include wheat, rice, maize, barley, sorghum, oats, rye, millet, soybeans, peanuts, beans, rapeseed, canola, sunflower, sugar cane, potatoes, sweet potatoes, cassava, sugar beets, tomatoes, plantains and bananas, and alfalfa.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EXPERIMENTAL

### General Materials and Methods

### Chemicals

5-Phenyl-2,4-pentadienoic acid (98%), phenylpropynoic acid (98%), phthalic anhydride (99%), triethylamine (99%), acetic anhydride (99%), ethylenediamine (99%), pyridinium chlorochromate (98%), dichloromethane (DCM, >99.9%), n-butanol (99%), and 1-Ethyl-(3′-dimethylaminopropyl)-carbodiimide hydrochloride (EDC, 98%) were purchased from Alfa Aesar (Ward Hill, Mass.). 3-(Trifluoromethyl) cinnamic acid (97%) was purchased from Oakwood Products, Inc. (West Columbia, S.C.). (9Z)-hexadec-9-enoic acid (>99%) and palmitoleyl alcohol were purchased from NU-CHEK PREP, Inc (Elysian, Minn.). Phe-O^{t}Bu.HCl was purchased from BACHEM (Torrance, Calif.). Gly-O^{t}Bu.HCl was purchased from CHEM-IMPEX International, Inc (Wood Dale, Ill.). 5-tert-Butyl 1-methyl glutamate HCl salt (>95%), N,N-dimethylaminopyridine (99%), methyl 2-(triphenylphosphoranylidene)acetate (98%) were purchased from Aldrich (Milwaukee, Wis.). Deuterated dimethyl sulfoxide (99.9% D) was purchased from Cambridge Isotope Laboratory, Inc (Andover, Mass.). Trifluoroacetic acid (TFA, 99.5%), Tetrahydrofuran (THF, ACS grade) and N,N-dimethylformamide (DMF, ACS grade) were purchased from EMD (Gibbstown, N.J.). D-glucosamine hydrochloride was purchased from Varsal, Inc (Warminster, Pa.). Methyl 2-amino-2-deoxy-β-D-glucopyranosyl-(1→4)-2-actemido-2-deoxy-β-D-glucopyranosyl-(1→4)-2-acetamido-2-deoxy-β-D-glucopyranosyl-(1→4)-2-acetamido-2-deoxy-β-D-glucopyranoside and ethylenediamine derivatized Merrified resin were prepared as described in U.S. Pat. No. 7,485,718, herein incorporated by reference.

### Seed Germination Assay

Materials were all sterilized before use. An aqueous solution of the test compound (25 mL, 10⁻⁷ M in DI-water) was prepared for a set of five repeat experiments. Five Petri dishes and 100 soybean seeds were used to test one compound. A piece of Whatman filter paper was used to cover the inner side of each Petri dish to allow uniform distribution of testing solution.

Twenty soybean seeds were placed on the filter paper area of one Petri dish. Five mL of the test compound solution was carefully poured in the Petri dish. Control experiments were set up the same way with 20 soybean seeds and 5 mL of DI-water per dish without any compounds. The lid was placed on the Petri dish and sealed with Parafilm. Five dishes with repeat experiments were stacked. One stack of dishes was wrapped twice with aluminum foil to prevent the seeds from receiving any light. The stacks were transferred to an incubator maintained at room temperature and the seeds were germinated in the dark.

After 20 h, the stacks were pulled out for measurement. The number of germinated seeds was counted and the percent germination on each dish was calculated. Radicle emergence was used as the germination indicator. The dishes were placed unwrapped at room temperature for one day and the number of germinated seeds was counted to ensure seeds were viable and that the germination results were not caused by poor seed quality. Dishes in which over 90% of the seeds germinated were considered normal. The standard deviation of five repeats was calculated. Test results with a 10% or lower standard deviation were considered good. Compounds acting as plant performance enhancers should promote a statistically significant increase in average percentage of germination compared to the control.

### Plant Growth Assay

Seeds germinated for 24 h using the seed germination assay above were exposed to light for an additional 40 h. The radical length was measured and the percentage of germinating seeds with a radical length greater than 1.5 cm was determined.

### Example 1

### Preparation of methyl 2-amino-2-deoxy-β-D-glucopyranoside 2

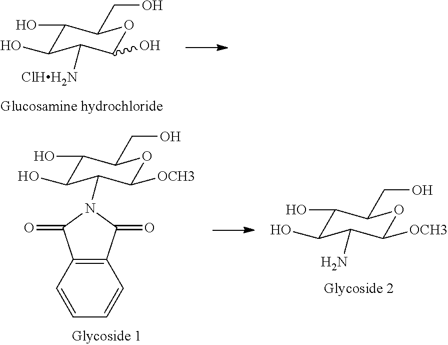

The title glycoside 2 was prepared in five steps starting from commercially available D-glucosamine hydrochloride. As described in U.S. Pat. No. 7,485,718, glucosamine hydrochloride was converted to glycoside 1. Then glycoside 1 was heated with ethylenediamine modified Merrifield resin in n-butanol at 110° C. to produce the desired product glycoside 2. ¹H NMR spectrum of the compound confirmed the identity of the structure.

### Example 2

### Synthesis of methyl 2-deoxy-2-({[(2E,11Z)-octadeca-2,11-dienoylamino]acetyl}amino)hexopyranoside 7

The synthesis of methyl 2-deoxy-2-N-({[(2E,11Z)-octadeca-2,11-dienoylamino]acetyl}amino)-β-D-glucopyranoside 7 was achieved by the following steps:

### a) Synthesis of (9Z)-hexadec-9-enal 3

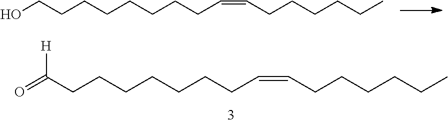

PCC (Pyridinium chlorochromate, 3.0 g, 13.4 mmol) and Celite (3.0 g) were added to a dry round bottom flask in a dry box. To this mixture, methylene chloride (50 g) was added. Palmitoleyl alcohol (2.0 g, 8.32 mmol) in methylene chloride (2 ml) was then added dropwise. The reaction mixture was stirred at room temperature for 4 h to completion as verified by TLC. Ethyl ether (18 ml) was added and the solution was vacuum filtered through a fritted funnel charged with 2 inches of silica gel and then washed with 100 ml of a hexane/ethyl acetate solution (9/1). The solution was pumped dry to give desired aldehyde 3 and used in the next step without further purification.

### b) Synthesis of methyl-(2E,11Z)-octadeca-2,11-dienoate 4

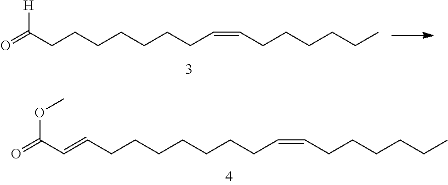 [SMILES: CCCCCC/C=C\CCCCCCCC=O ; CCCCCC/C=C\CCCCCCC/C=C/C(=O)OC]

Compound 3 was dissolved in CH₂Cl₂ (200 ml) and added with methyl 2-(triphenylphosphoranylidene)acetate (4.0 g, 12 mmol). The resulting mixture was stirred at room temperature overnight. Ethyl ether (18 mL) was added and the solution was vacuum filtered through a fritted funnel charged with 2 in of silica gel and washed with 100 ml of a hexane/ethyl acetate solution (9/1). The solution was then pumped dry. The crude product was purified by column chromatography to afford 2.2 g of desired compound 4 in 90% yield. The structure was characterized by ¹H NMR.

¹H NMR (500 MHz, CDCl₃): δ 6.97 (dt, J₁=15.6 Hz, J₂=7.0 Hz, 1H), 5.81 (dt, J₁=15.6 Hz, J₂=3.2 Hz, 1H), 5.38-5.31 (m, 2H), 3.72 (s, 3H), 2.21-2.17 (m, 2H), 2.10-1.99 (m, 4H), 1.48-1.42 (m, 2H), 1.36-1.26 (m, 16H), 0.88 (t, J=7.0 Hz, 3H).

### c) Synthesis of (2E,11Z)-octadeca-2,11-dienoic acid 5

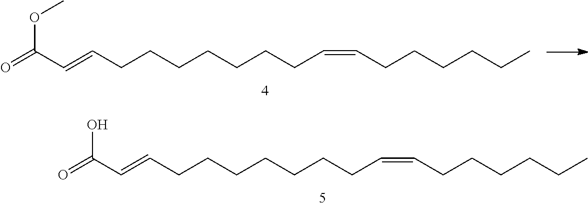 [SMILES: CCCCCC/C=C\CCCCCCC/C=C/C(=O)OC ; CCCCCC/C=C\CCCCCCC/C=C/C(=O)O]

Compound 4 (0.5 g, 1.7 mmol) synthesized from the above procedure was dissolved in a mixture of 1:1 MeOH/THF (total volume 5 ml) in a vial. LiOH aqueous solution (5 mL, 15 wt % in DI-water) was added and the mixture was stirred at room temperature for about 4 h. The reaction mixture was concentrated under vacuum. The resulting residue was diluted with water (5 ml), acidified with 2N HCl to pH 1-2 and extracted with diethyl ether (3 times, 20 ml/each). The combined organic extracts were washed with brine (15 ml) and water (15 ml), dried over anhydrous Na₂SO₄, filtered, and concentrated to give 0.47 g of the corresponding acid 5 quantitatively, which was used in next step without further purification.

### d) Synthesis of N-[(2E,11Z)-octadeca-2,11-dienoyl]glycine 6

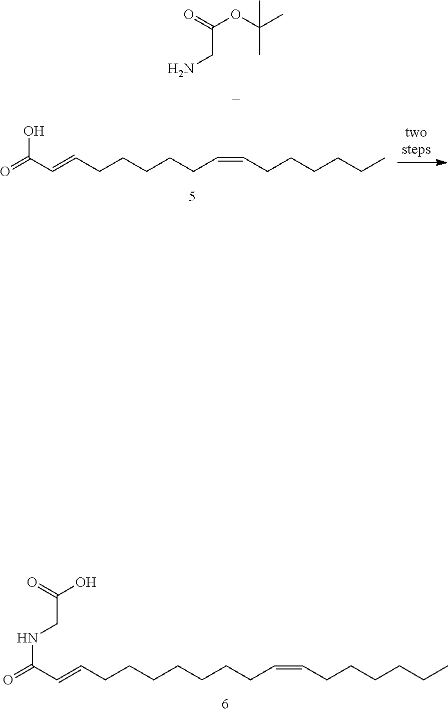

Compound 5 (0.2 g, 1.15 mmol) was dissolved in 1:2 DMF/THF (total volume is 5 ml). To this solution, 4-N,N-dimethylaminopyridine (DMAP) (0.25 g, 2.07 mmol), 1-Ethyl-3-(3′-dimethylaminopropyl)carbodiimide hydrochloride (EDC) (0.40 g, 2.07 mmol), and Gly-O^{t}Bu.HCl (0.23 g, 1.38 mmol) were added and the reaction was stirred at room temperature overnight. After completion of the reaction, the mixture was pumped dry and washed with DI-H₂O three times (5 ml/each). The product was purified by column chromatography on a column of silica gel to afford 220 mg of the homogeneous product in 67% yield. The structure was characterized by ¹H NMR: ¹H NMR (500 MHz, CDCl₃): δ 6.85 (dt, J₁=15.3 Hz, J₂=7.0 Hz, 1H), 5.92 (br, 1H), 5.81 (d, J=15.3 Hz, 1H), 5.35-5.33 (m, 2H), 4.0 (d, J=5.0 Hz, 2H), 2.19-2.14 (m, 2H), 2.02-1.99 (m, 4H), 1.47 (s, 9H), 1.46-1.40 (m, 2H), 1.33-1.25 (m, 16H), 0.88 (t, J=6.9 Hz, 3H).

The resulting t-butyl ester from above was dissolved in 1:1 DCM/TFA solution (total volume 1.5 ml). The mixture was stirred at room temperature for 2 h to obtain the free acid 6 form for the next step of the synthesis. The structure was characterized by ¹H NMR.

¹H NMR (500 MHz, CDCl₃): ¹H NMR (500 MHz, CDCl₃): δ 6.9-6.87 (m, 1H), 6.41 (br, 1H), 5.86 (d, J=15.3 Hz, 1H), 5.38-5.33 (m, 1H), 5.07-5.02 (m, 1H), 4.13-4.12 (m, 2H), 2.20-2.16 (m, 2H), 2.02-1.94 (m, 2H), 1.66-1.58 (m, 2H), 1.44-1.42 (m, 2H), 1.28-1.27 (m, 16H), 0.87 (t, J=6.7 Hz, 3H).

### e) Synthesis of methyl 2-deoxy-2-({[(2E,11Z)-octadeca-2,11-dienoylamino]acetyl}amino)hexopyranoside 7

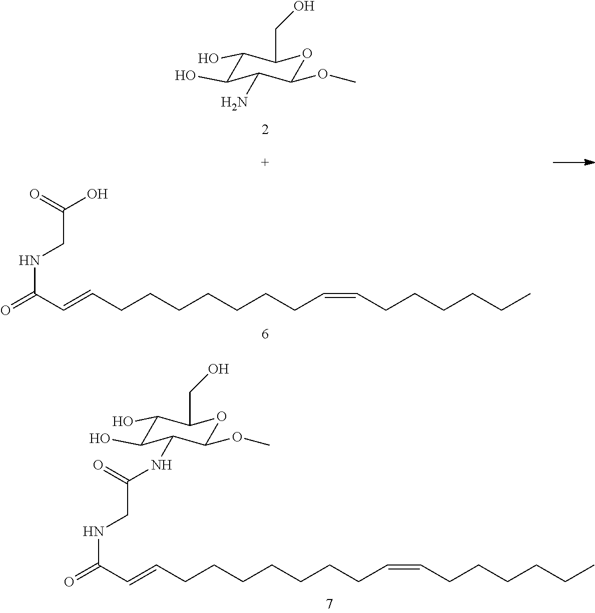 [SMILES: COC1OC(CO)C(O)C(O)C1N ; CCCCCC/C=C\CCCCCCC/C=C/C(=O)NCC(=O)O ; CCCCCC/C=C\CCCCCCC/C=C/C(=O)NCC(=O)NC1C(OC)OC(CO)C(O)C1O]

Compound 6 (0.04 g, 0.1 mmol) was dissolved in a mixture of 1:2 DMF/THF (total volume 1 ml). To this solution, DMAP (0.02 g, 0.12 mmol), EDC (0.02 g, 0.12 mmol), and compound 2 (0.04 g, 0.21 mmol) were added and the reaction was stirred at room temperature overnight. The reaction mixture was pumped dry and washed with DI-H₂O three times (1 ml/each). The final product was purified with column chromatography 7 to provide 50 mg of desired product in 36% yield. The structure was characterized by ¹H NMR and LC-MS.

¹H NMR (500 MHz, DMSO-D₆): δ 8.06 (t, J=5.7 Hz, 1H), 7.76 (d, J=9.1 Hz, 1H), 6.62 (dt, J₁=15.4 Hz, J₂=7.0 Hz, 1H), 6.00 (d, J=15.4 Hz, 1H), 5.33-5.31 (m, 2H), 5.01 (br, 1H), 4.89 (br, 1H), 4.57-4.55 (m, 1H), 4.20 (d, J=8.5 Hz, 1H), 3.79-3.67 (m, 4H), 3.31 (s, 3H), 3.01 (br, 2H), 2.14-2.09 (m, 2H), 2.00-1.96 (m, 4H), 1.39-1.35 (m, 2H), 1.29-1.24 (m, 16H), 0.85 (t, J=6.8 Hz, 3H). LC-MS (ESI): m/z 513 [M+1]⁺.

### Example 2A

### Testing of Soybean Seeds Treated with Compound methyl 2-deoxy-2-({[(2E,11Z)-octadeca-2,11-dienoylamino]acetyl}amino)hexopyranoside 7

Compound 7 prepared in Example 2 was evaluated using the seed germination assay described in General Material & Methods. Soybean seeds treated with this compound showed 46% germination at 20 h with a standard deviation of 6%. Control soybean seeds showed 24% germination at 20 hours with a standard deviation of 4%.

The same compound was evaluated using the plant growth assay described Example 1. Seventy-nine percent (79%) of the germinated soybean seeds treated with this compound exhibited radical lengths greater than 1.5 cm, with a standard deviation of 6%. Fifty-five percent (55%) of the germinated control soybean seeds exhibited radical lengths greater than 1.5 cm, with a standard deviation of 9%.

### Example 3

### Synthesis of Methyl 2-deoxy-2-N-({[(9Z)-hexadec-9-enoylamino]acetyl}amino)-β-D-glucopyranoside 9

(9Z)-Hexadec-9-enoic acid (1.5 g, 5.91 mmol) was dissolved in 1:2 DMF/THF (total volume 10 ml). To this solution, DMAP (0.87 g, 7.09 mmol), EDC (1.36 g, 7.09 mmol), and Gly-O^{t}Bu.HCl (1.98 g, 11.81 mmol) was added and the reaction was stirred at room temperature overnight. The reaction is represented in the following scheme:

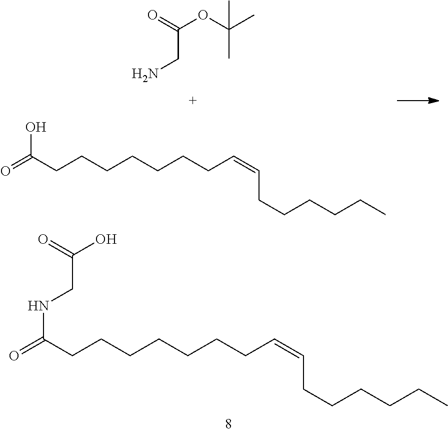

After completion of the reaction, the mixture was pumped dry and washed with DI-H₂O three times (5 ml/each). The final product was purified with column chromatography to provide 1060 mg of the desired product in 49% yield. The structure was characterized by ¹H NMR.

¹H NMR (500 MHz, CDCl₃): δ 8.11 (t, J=6.0 Hz, 1H), 5.34-5.30 (m, 2H), 3.67 (d, J=6.0 Hz, 2H), 2.09 (t, J=7.4 Hz, 2H), 2.00-1.96 (m, 4H), 1.51-1.46 (m, 2H), 1.39 (s, 9H), 1.30-1.24 (m, 16H), 0.85 (t, J=6.9 Hz, 3H).

The resulting t-butyl ester was dissolved in 1:1 DCM/TFA solution (total volume 1.5 mL). The mixture was stirred at room temperature for 2 h to obtain the free acid form 8 for the next step of the synthesis. The reaction is represented in the following scheme:

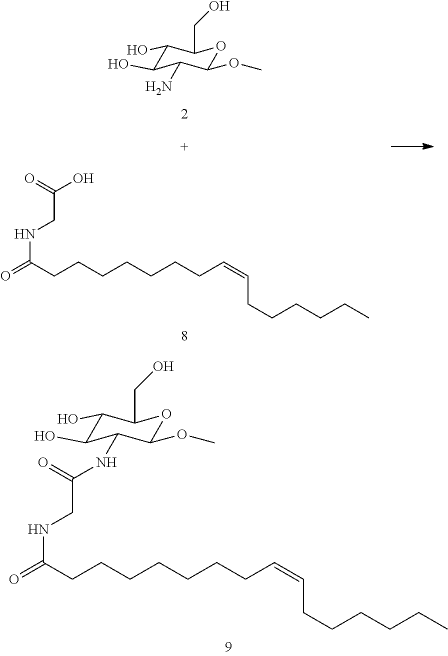 [SMILES: COC1OC(CO)C(O)C(O)C1N ; CCCCCC/C=C\CCCCCCCC(=O)NCC(=O)O ; CCCCCC/C=C\CCCCCCCC(=O)NCC(=O)NC1C(OC)OC(CO)C(O)C1O]

Compound 8 (0.2 g, 0.65 mmol) was dissolved in 1:2 DMF/THF (total volume is 5 ml). To this solution, DMAP (0.09 g, 0.71 mmol), EDC (0.14 g, 0.71 mmol), and compound 2 (0.22 g, 1.19 mmol) were added and the reaction was stirred at room temperature overnight. The reaction mixture was pumped dry and washed with DI-H₂O three times (1 mL/each). The final product was purified with column chromatography to provide 96 mg of desired product 9 in 30% yield. The structure was characterized by LC-MS and ¹H NMR.

¹H NMR (500 MHz, DMSO-D⁶): δ 7.91 (t, J=5.6 Hz, 1H), 7.69 (d, J=9.1 Hz, 2H), 7.37 (t, J=9.2 Hz, 2H), 5.35-5.29 (m, 2H), 5.01 (br, 1H), 4.88 (br, 1H), 4.57-4.55 (m, 1H), 4.20 (d, J=8.5 Hz, 1H), 3.74-3.62 (m, 4H), 3.31 (s, 3H), 3.11-3.05 (m, 2H), 2.11 (t, J=7.5 Hz, 2H), 2.00-1.96 (m, 4H), 1.48-1.46 (m, 2H), 1.29-1.24 (m, 16H), 0.85 (t, J=6.8 Hz, 3H). LC-MS (ESI): m/z 487 [M+1]⁺.

### Example 3A

### Testing of Soybean Seeds Treated with Methyl 2-deoxy-2-N-({[(9Z)-hexadec-9-enoylamino]acetyl}amino)-β-D-glucopyranoside 9

Compound 9 prepared in Example 3 was evaluated using the seed germination assay described in General Material & Methods. Soybean seeds treated with this compound showed 33% germination at 20 hours with a standard deviation of 3%. Control soybean seeds showed 24% germination at 20 hours with a standard deviation of 4%.

The same compound was evaluated using the plant growth assay described Example 1. Seventy-three percent (73%) of the germinated soybean seeds treated with this compound exhibited radical lengths greater than 1.5 cm, with a standard deviation of 6%. Fifty-five percent (55%) of the control soybean seeds exhibited radical lengths greater than 1.5 cm, with a standard deviation of 9%.

### Example 4

### Synthesis of methyl 2-deoxy-2-[({[(2E,4E)-5-phenylpenta-2,4-dienoyl]amino}acetyl)amino]-β-D-glucopyranoside 11

5-Phenylpenta-2,4-dienoic acid (0.2 g, 1.15 mmol) was dissolved in 1:2 DMF/THF (total volume is 5 ml). To this solution, DMAP (0.25 g, 2.07 mmol), EDC (0.4 g, 2.07 mmol), and Gly-O^{t}Bu.HCl (0.23 g, 1.38 mmol) were added and the reaction was stirred at room temperature overnight. The reaction is represented in the following scheme:

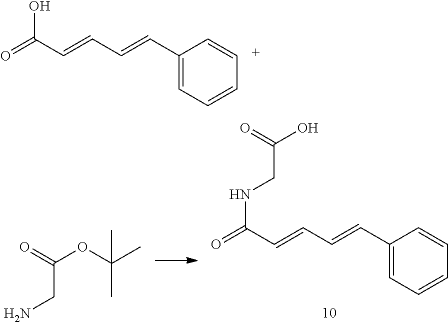

After completion of the reaction, the mixture was pumped dry and washed with DI-H₂O three times (5 ml/each). The final product was purified with column chromatography to provide 220 mg of the desired product in 67% yield. The structure was characterized by ¹H NMR.

¹H NMR (500 MHz, DMSO-D⁶): δ 8.42 (t, J=6.0 Hz, 1H), 7.56 (d, J=7.4 Hz, 2H), 7.37 (t, J=7.4 Hz, 2H), 7.30 (t, J=7.4 Hz, 1H), 7.25-7.20 (m, 1H), 7.10-7.03 (m, 1H), 6.96 (d, J=15.6 Hz, 1H), 6.22 (d, J=15.0 Hz, 1H), 3.82 (d, J=6.0 Hz, 2H), 1.41 (s, 9H).

The resulting t-butyl ester (0.1 g, 0.35 mmol) was dissolved in 1:1 DCM/TFA (total volume is 1.5 mL). The mixture was stirred at room temperature for 2 h to obtain compound 10 for the next step of the synthesis. The structure was characterized by ¹H NMR and LC-MS.

¹H NMR (500 MHz, DMSO-D⁶): δ 8.42 (t, J=6.0 Hz, 1H), 7.56 (d, J=7.4 Hz, 2H), 7.37 (t, J=7.4 Hz, 2H), 7.30 (t, J=7.4 Hz, 1H), 7.25-7.20 (m, 1H), 7.09-7.04 (m, 1H), 6.96 (d, J=15.6 Hz, 1H), 6.22 (d, J=15.0 Hz, 1H), 3.85 (d, J=6.0 Hz, 2H).

The above generated compound 10 was dissolved in a mixture of 1:2 DMF/THF (total volume is 3 ml). To this solution, DMAP (0.08 g, 0.63 mmol), EDC (0.12 g, 0.63 mmol), and compound 2 (0.08 g, 0.42 mmol) were added and the reaction was stirred at room temperature overnight. The reaction is represented in the following scheme:

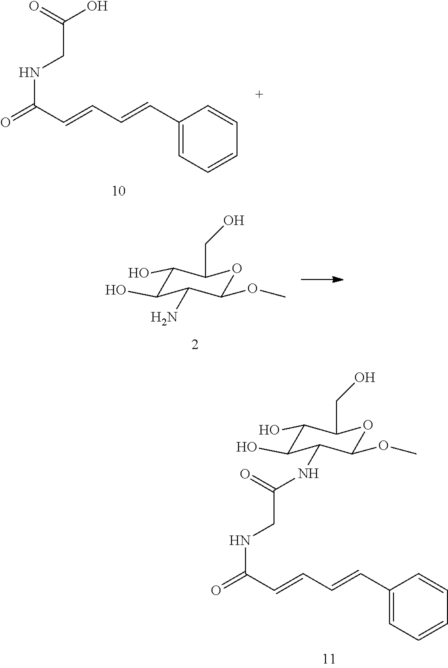 [SMILES: O=C(O)CNC(=O)/C=C/C=C/c1ccccc1 ; COC1OC(CO)C(O)C(O)C1N ; COC1OC(CO)C(O)C(O)C1NC(=O)CNC(=O)/C=C/C=C/c1ccccc1]

The reaction mixture was then pumped dry and washed with DI-H₂O three times (1 mL/each). The final product was purified with column chromatography to afford 21 mg of desired product 11 in 41% yield. The structure was characterized by ¹H NMR.

¹H NMR (500 MHz, DMSO-D⁶): δ 8.28 (t, J=5.8 Hz, 1H), 7.79 (d, J=9.1 Hz, 1H), 7.56 (d, J=7.4 Hz, 2H), 7.37 (t, J=7.4 Hz, 2H), 7.30 (t, J=7.4 Hz, 1H), 7.24-7.19 (m, 1H), 7.08-7.03 (m, 1H), 6.96 (d, J=15.6 Hz, 1H), 6.27 (d, J=15.0 Hz, 1H), 5.02 (d, J=4.9 Hz, 1H), 4.91 (d, J=5.2 Hz, 1H), 4.58 (t, J=5.9 Hz, 1H), 4.21 (d, J=8.5 Hz, 1H), 3.87-3.77 (m, 2H), 3.71-3.67 (m, 1H), 3.32 (s, 3H), 3.10-3.08 (m, 2H). LC-MS (ESI): m/z 407 [M+1]⁺.

### Example 4A

### Testing of Soybean Seeds Treated with methyl 2-deoxy-2-[({[(2E,4E)-5-phenylpenta-2,4-dienoyl]amino}acetyl)amino]-β-D-glucopyranoside 11

Compound 11 in Example 4 was evaluated using the seed germination assay described in General Material & Methods. Soybean seeds treated with this compound showed 39% germination at 20 hours with a standard deviation of 4%. Control soybean seeds showed 24% germination at 20 hours with a standard deviation of 4%.

The same compound was evaluated using the plant growth assay described Example 1. Eighty-two percent (82%) of the germinated soybean seeds treated with this compound exhibited radical lengths greater than 1.5 cm, with a standard deviation of 3%. Fifty-five percent (55%) of the control soybean seeds exhibited radical lengths greater than 1.5 cm, with a standard deviation of 9%.

### Example 5

### Synthesis of Methyl 2-deoxy-2-({N-[(2E,4E)-5-phenylpenta-2,4-dienoyl]phenylalanyl}amino)-β-D-glucopyranoside 13

5-Phenylpenta-2,4-dienoic acid (0.2 g, 1.15 mmol) was dissolved in 1:2 DMF/THF (total volume is 5 ml). To this solution, DMAP (0.25 g, 2.07 mmol), EDC (0.4 g, 2.07 mmol), and Phe-O^{t}Bu.HCl (0.36 g, 1.38 mmol) were added and the reaction was stirred at room temperature overnight. The reaction is represented in the following scheme:

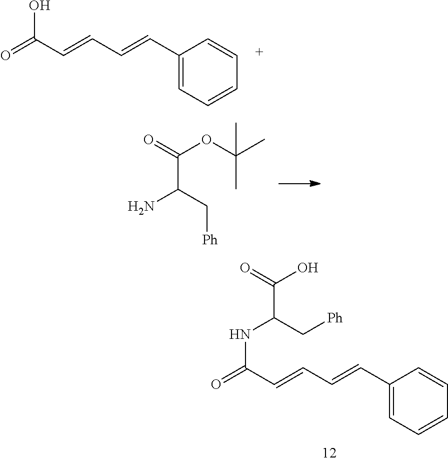

After completion of the reaction, the mixture was pumped dry and washed with DI-H₂O three times (1 ml/each). The final product was purified with column chromatography to provide 310 mg of the desired product in 72% yield. The structure was characterized by ¹H NMR.

¹H NMR (500 MHz, DMSO-D⁶): δ 8.47 (t, J=7.8 Hz, 1H), 7.53 (d, J=7.4 Hz, 2H), 7.36 (t, J=7.4 Hz, 2H), 7.31-7.25 (m, 3H), 7.22-7.14 (m, 4H), 7.04-6.98 (m, 1H), 6.93 (d, J=15.6 Hz, 1H), 6.20 (d, J=15.0 Hz, 1H), 4.47-4.42 (m, 1H), 3.00-2.88 (m, 2H), 1.30 (s, 9H).

The resulting t-butyl ester (0.1 g, 0.26 mmol) was dissolved in 1:1 DCM/TFA solution (total volume is 1.5 mL). The mixture was stirred at room temperature for 2 h to obtain compound 12 for the next step of the synthesis. The reaction is represented by the following scheme:

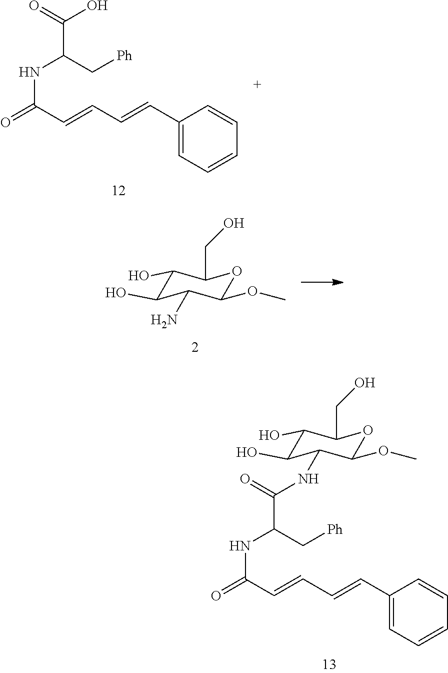 [SMILES: O=C(/C=C/C=C/c1ccccc1)NC(Cc1ccccc1)C(=O)O ; COC1OC(CO)C(O)C(O)C1N ; COC1OC(CO)C(O)C(O)C1NC(=O)C(Cc1ccccc1)NC(=O)/C=C/C=C/c1ccccc1]

The above generated compound 12 was dissolved in 1:2 DMF/THF (total volume is 3 ml). To this solution, DMAP (0.05 g, 0.39 mmol), EDC (0.07 g, 0.39 mmol), and compound 2 (0.06 g, 0.31 mmol) were added and the reaction was stirred at room temperature overnight. Then the reaction mixture was pumped dry and washed with DI-H₂O three times (1 mL/each). The final product was purified with column chromatography to provide 58 mg of desired product 13 in 45% yield. The structure was characterized by ¹H NMR and LC-MS.

¹H NMR (500 MHz, DMSO-D⁶): δ 8.26 (t, J=8.6 Hz, 1H), 7.99 (t, J=10.1 Hz, 1H), 7.54 (d, J=7.4 Hz, 2H), 7.36 (t, J=7.4 Hz, 2H), 7.31-7.23 (m, 5H), 7.18-7.09 (m, 2H), 7.04-6.99 (m, 1H), 6.91 (d, J=15.4 Hz, 1H), 6.24-6.20 (m, 1H), 5.02-4.98 (m, 1H), 4.90-4.84 (m, 1H), 4.65-4.60 (m, 1H), 4.55-4.52 (m, 1H), 4.27-4.18 (m, 1H), 3.72-3.68 (m, 1H), 3.52-3.42 (m, 2H), 3.31 (s, 3H), 3.10-3.09 (m, 2H), 3.07-3.02 (m, 1H), 2.82-2.76 (m, 1H). LC-MS (ESI): m/z 497 [M+1]⁺.

### Example 5A

### Testing of Soybean Seeds Treated with Methyl 2-deoxy-2-({N-[(2E,4E)-5-phenylpenta-2,4-dienoyl]phenylalanyl}amino)-β-D-glucopyranoside 13

Compound 13 prepared in Example 5 was evaluated using the seed germination assay described in General Material & Methods. Soybean seeds treated with this compound showed 32% germination at 20 hours with a standard deviation of 5%. Control soybean seeds showed 24% germination in 20 hours with a standard deviation of 4%.

The same compound was evaluated using the plant growth assay described Example 1. Eighty percent (80%) of the germinated soybean seeds treated with this compound exhibited radical lengths greater than 1.5 cm, with a standard deviation of 7%. Fifty-five percent (55%) of the control soybean seeds exhibited radical lengths greater than 1.5 cm, with a standard deviation of 9%.

### Example 6

### Synthesis of methyl 5-{[4,5-dihydroxy-6-(hydroxymethyl)-2-methoxytetrahydro-2H-pyran-3-yl]amino}-5-oxo-2-{[(2E,4E)-5-phenylpenta-2,4-dienoyl]amino}pentanoate 15

5-Phenylpenta-2,4-dienoic acid (0.3 g, 1.72 mmol) was dissolved in 1:2 DMF/THF (total volume is 5 ml). To this solution, DMAP (0.32 g, 2.58 mmol), EDC (0.5 g, 2.58 mmol), and 5-tert-butyl 1-methyl glutamate HCl salt (0.50 g, 2.58 mmol) was added and the reaction was stirred at room temperature overnight. The reaction is represented by the following scheme:

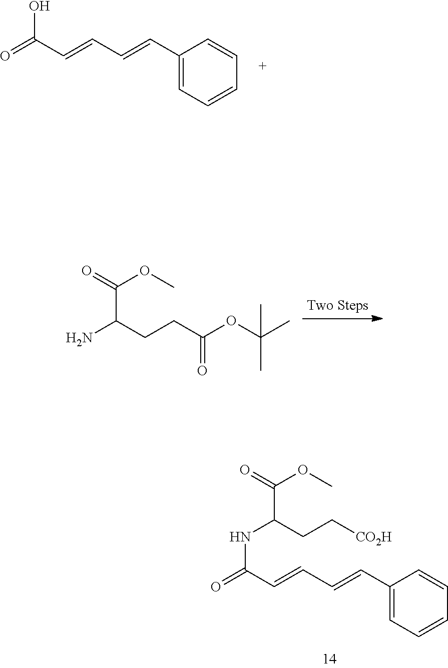 [SMILES: O=C(O)/C=C/C=C/c1ccccc1 ; COC(=O)C(N)CCC(=O)OC(C)(C)C ; COC(=O)C(CCC(=O)O)NC(=O)/C=C/C=C/c1ccccc1]

After completion of the reaction, the mixture was pumped dry and washed with DI-H₂O three times (1 ml/each). The final product was purified with column chromatography to provide 72 mg of the desired product in 35% yield. The structure was characterized by ¹H NMR.

¹H NMR (500 MHz, DMSO-D⁶): δ 8.49 (t, J=7.6 Hz, 1H), 7.54 (d, J=7.4 Hz, 2H), 7.36 (t, J=7.4 Hz, 2H), 7.30 (t, J=7.4 Hz, 1H), 7.24-7.19 (m, 1H), 7.06-7.01 (m, 1H), 6.96 (d, J=15.6 Hz, 1H), 6.21 (d, J=15.0 Hz, 1H), 4.37-4.33 (m, 1H), 3.62 (s, 3H), 2.32-2.25 (m, 2H), 2.00-1.93 (m, 1H), 1.85-1.78 (m, 1H), 1.37 (s, 9H). The resulting t-butyl ester was dissolved in 1:1 DCM/TFA (total volume 1.5 ml). The mixture was stirred at room temperature for 2 h to obtain compound 14 for the next step of the synthesis.

The above generated compound 14 (0.1 g, 0.32 mmol) was dissolved in a mixture of 1:2 DMF/THF (total volume is 2 ml). To this solution, DMAP (0.05 g, 0.39 mmol), EDC (0.09 g, 0.47 mmol), and compound 2 (0.07 g, 0.35 mmol) were added and the reaction was stirred at room temperature overnight. The reaction is represented by the following scheme:

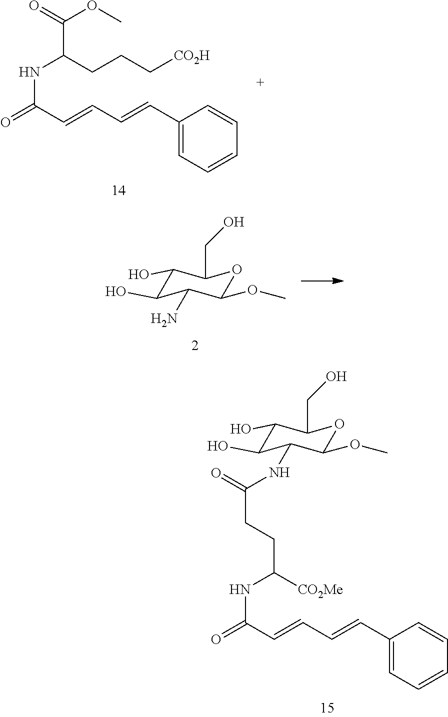 [SMILES: COC(=O)C(CCCC(=O)O)NC(=O)/C=C/C=C/c1ccccc1 ; COC1OC(CO)C(O)C(O)C1N ; COC(=O)C(CCC(=O)NC1C(OC)OC(CO)C(O)C1O)NC(=O)/C=C/C=C/c1ccccc1]

The reaction mixture was then pumped dry and washed with DI-H₂O three times (1 ml/each). The final product was purified with column chromatography to provide 65 mg of desired product 15 in 42% yield. The structure was characterized by LC-MS and ¹H NMR.

¹H NMR (500 MHz, DMSO-D⁶): δ 8.53 (d, J=7.2 Hz, 1H), 7.74 (d, J=9.0 Hz, 1H), 7.55 (d, J=7.4 Hz, 2H), 7.36 (t, J=7.4 Hz, 2H), 7.30 (t, J=7.4 Hz, 1H), 7.24-7.18 (m, 1H), 7.09-7.01 (m, 1H), 6.96 (d, J=15.6 Hz, 1H), 6.21 (d, J=15.0 Hz, 1H), 5.09-5.08 (m, 1H), 4.96-4.94 (m, 1H), 4.70-4.68 (m, 1H), 4.35-4.31 (m, 1H), 4.17 (d, J=8.4 Hz, 1H), 3.63 (s, 3H), 3.46-3.38 (m, 2H), 3.30 (s, 3H), 3.10-3.03 (m, 2H), 2.17-2.14 (m, 2H), 2.02-1.97 (m, 1H), 1.86-1.79 (m, 1H). LC-MS (ESI): m/z 493 [M+1]⁺.

### Example 6A

### Testing of Soybean Seeds Treated with methyl 2-deoxy-2-({N-[(2E,4E)-5-phenylpenta-2,4-dienoyl]phenylalanyl}amino)-β-D-glucopyranoside 15

Compound 15 prepared in Example 6 was evaluated using the seed germination assay described in General Material & Methods. Soybean seeds treated with this compound showed 31% germination at 20 hours with a standard deviation of 4%. Control soybean seeds showed 24% germination at 20 hours with a standard deviation of 4%.

The same compound was evaluated using the plant growth assay described Example 1. Seventy-four percent (74%) of the germinated soybean seeds treated with this compound exhibited radical lengths greater than 1.5 cm, with a standard deviation of 9%. Fifty-five percent (55%) of the control soybean seeds exhibited radical lengths greater than 1.5 cm, with a standard deviation of 9%.

### Example 7

### Synthesis of methyl 2-deoxy-2-{[({(2E)-3-[3-(trifluoromethyl)phenyl]prop-2-enoyl}amino)acetyl]amino}-β-D-glucopyranoside 17

3-(Trifluoromethyl)cinnamic acid (0.2 g, 0.93 mmol) was dissolved in 1:2 DMF/THF (total volume 4 ml). To this solution, DMAP (0.17 g, 1.4 mmol), EDC (0.27 g, 1.4 mmol), and Gly-O^{t}Bu.HCl (0.23 g, 1.38 mmol) was added and the reaction was stirred at room temperature overnight. The reaction is represented by the following scheme:

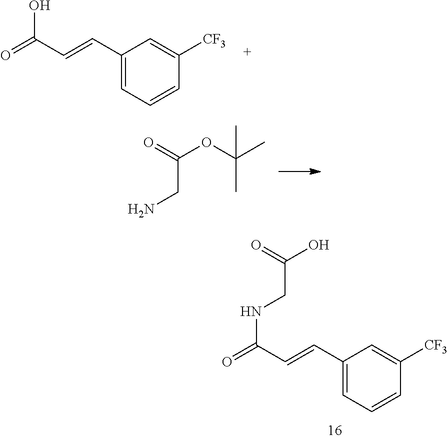

After completion of the reaction, the mixture was pumped dry and washed with DI-H₂O three times (5 ml/each). The final product was purified with column chromatography to provide 210 mg of the desired product in 69% yield. The structure was characterized by ¹H NMR.

¹H NMR (500 MHz, DMSO-D⁶): δ 8.45 (t, J=6.0 Hz, 1H), 7.93 (s, 1H), 7.89 (d, J=7.8 Hz, 1H), 7.72 (d, J=7.8 Hz, 1H), 7.65 (t, J=7.8 Hz, 1H), 7.54 (d, J=15.9 Hz, 1H), 6.87 (d, J=15.9 Hz, 1H), 3.87 (d, J=6.0 Hz, 2H), 1.41 (s, 9H). The resulting t-butyl ester (0.1 g) was dissolved in 1:1 DCM/TFA solution (DCM/TFA (total volume 1.5 ml). The mixture was stirred at room temperature for 2 h to obtain compound 16 for the next step of the synthesis.

The above generated compound 16 was dissolved in a mixture of 1:2 DMF/THF (total volume 2 ml). To this solution, DMAP (0.05 g, 0.45 mmol), EDC (0.09 g, 0.45 mmol), and compound 2 (0.06 g, 0.33 mmol) were added and the reaction was stirred at room temperature overnight. The reaction is represented by the following scheme:

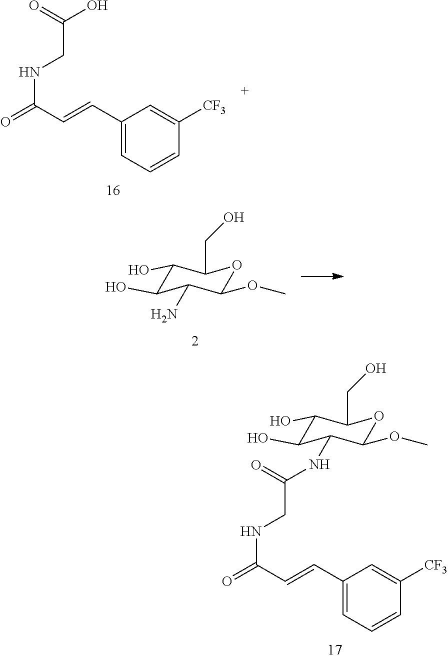 [SMILES: O=C(O)CNC(=O)/C=C/c1cccc(C(F)(F)F)c1 ; COC1OC(CO)C(O)C(O)C1N ; COC1OC(CO)C(O)C(O)C1NC(=O)CNC(=O)/C=C/c1cccc(C(F)(F)F)c1]

The reaction mixture then was pumped dry and washed with DI-H₂O three times (1 ml/each). The final product was purified with column chromatography to provide 85 mg of desired product 17 in 62% yield. The structure was characterized by ¹H NMR.

¹H NMR (500 MHz, DMSO-D⁶): δ 8.32 (t, J=5.6 Hz, 1H), 7.92 (s, 1H), 7.92-7.85 (m, 2H), 7.72 (d, J=7.8 Hz, 1H), 7.65 (d, J=7.8 Hz, 1H), 7.51 (t, J=15.8 Hz, 1H), 6.90 (d, J=15.8 Hz, 1H), 5.09-5.08 (m, 1H), 4.99-4.98 (m, 1H), 4.68-4.66 (m, 1H), 4.21 (d, J=8.5 Hz, 1H), 3.91-3.80 (m, 4H), 3.70 (s, 2H), 3.32 (s, 3H), 3.10-3.07 (m, 2H).

### Example 7A

### Testing of Soybean Seeds Treated with Methyl 2-deoxy-2-{[({(2E)-3-[3-(trifluoromethyl)phenyl]prop-2-enoyl}amino)acetyl]amino}-β-D-glucopyranoside 17

Compound 17 prepared in Example 7 was evaluated using the seed germination assay described in General Material & Methods. Soybean seeds treated with this compound showed 44% germination at 20 hours with a standard deviation of 6%. Control soybean seeds showed 24% germination at 20 hours with a standard deviation of 4%.

The same compound was evaluated using the plant growth assay described Example 1. Eighty-six percent (86%) of the germinated soybean seeds treated with this compound exhibited radical lengths greater than 1.5 cm, with a standard deviation of 7%. Fifty-five percent (55%) of the control soybean seeds exhibited radical lengths greater than 1.5 cm, with a standard deviation of 9%.

### Example 8

### Synthesis of Methyl 2-deoxy-2-N-({[(3-phenylprop-2-ynoyl)amino]acetyl}amino)-β-D-glucopyranoside 19

Phenylpropyoic acid (0.5 g, 3.42 mmol) was dissolved in 1:2 DMF/THF (total volume 15 ml). To this solution, DMAP (0.63 g, 5.13 mmol), EDC (0.98 g, 5.13 mmol), and Gly-O^{t}Bu.HCl (0.86 g, 5.13 mmol) were added and the reaction was stirred at room temperature overnight. The reaction is represented by the following scheme:

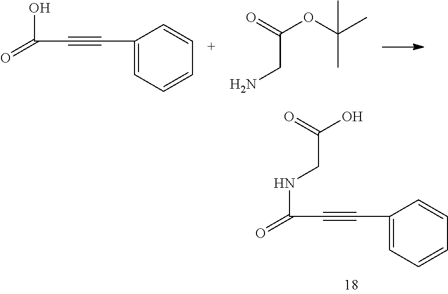

After completion of the reaction, the mixture was pumped dry and washed with DI-H₂O three times (5 ml/each). The final product was purified with column chromatography to provide 480 mg of the desired product in 54% yield. The structure was characterized by ¹H NMR (500 MHz, DMSO-D⁶): δ 9.11 (t, J=6.0 Hz, 1H), 7.67-7.56 (m, 2H), 7.54-7.45 (m, 3H), 3.80 (d, J=6.0 Hz, 2H), 1.41 (s, 9H). The resulting t-butyl ester (0.48 g, 1.85 mmol) was dissolved in 1:1 DCM/TFA (total volume 2.5 mL). The mixture was stirred at room temperature for 2 h to yield compound 18 for the next step of the synthesis. The structure was characterized by ¹H NMR.

¹H NMR (500 MHz, DMSO-D⁶): δ 9.11 (t, J=6.0 Hz, 1H), 7.59-7.56 (m, 2H), 7.52-7.49 (m, 1H), 7.47-7.43 (m, 2H), 3.82 (d, J=6.0 Hz, 2H).

The above generated compound 18 (0.3 g, 1.48 mmol) was dissolved in a mixture of 1:2 DMF/THF (total volume 15 ml). To this solution, DMAP (0.271 g, 2.22 mmol), EDC (0.42 g, 2.22 mmol), and compound 2 (0.31 g, 1.62 mmol) were added and the reaction was stirred at room temperature overnight. The reaction is represented by the following scheme:

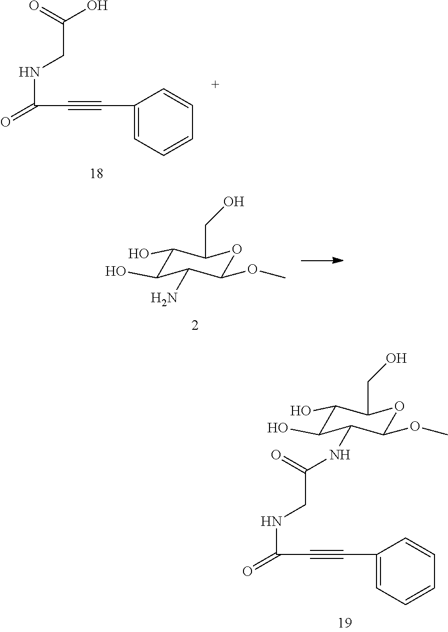 [SMILES: O=C(O)CNC(=O)C#Cc1ccccc1 ; COC1OC(CO)C(O)C(O)C1N ; COC1OC(CO)C(O)C(O)C1NC(=O)CNC(=O)C#Cc1ccccc1]

The reaction mixture was then pumped dry and washed with DI-H₂O three times (1 ml/each). The final product was purified with column chromatography to provide 310 mg of desired product 19 in 56% yield. The structure was characterized by LC-MS and ¹H NMR.

¹H NMR (500 MHz, DMSO-D⁶): δ 8.92 (t, J=6.0 Hz, 1H), 7.85 (d, J=9.1 Hz, 1H), 7.58-7.56 (m, 2H), 7.52-749 (m, 1H), 7.47-7.44 (m, 2H), 5.11 (d, J=4.9 Hz, 1H), 4.99 (d, J=5.2 Hz, 1H), 4.71 (t, J=6.0 Hz, 1H), 4.21 (d, J=8.4 Hz, 1H), 3.86-3.76 (m, 4H), 3.48-3.41 (m, 2H), 3.31 (s, 3H), 3.12-3.05 (m, 2H). LC-MS (ESI): m/z 379 [M+1]⁺.

### Example 8A

### Testing of Soybean Seeds Treated with Methyl 2-deoxy-2-N-({[(3-phenylprop-2-ynoyl)amino]acetyl}amino)-β-D-glucopyranoside 19

Compound 19 prepared in Example 8 was evaluated using the seed germination assay described in General Material & Methods. Soybean seeds treated with this compound showed 30% germination at 20 hours with a standard deviation of 2%. Control soybean seeds showed 24% germination at 20 hours with a standard deviation of 4%.

The same compound was evaluated using the plant growth assay described Example 1. Seventy-one percent (71%) of the germinated soybean seeds treated with this compound exhibited radical lengths greater than 1.5 cm, with a standard deviation of 8%. Fifty-five percent (55%) of the control soybean seeds exhibited radical lengths greater than 1.5 cm, with a standard deviation of 9%.

### Example 9

### The synthesis of Methyl 2-deoxy-2-N-({[(2E,11E)-octadeca-2,11-dienoylamino]acetyl}amino)β-D-glucopyranosyl(1→4)-2-acetamido-2-deoxy-β-D-glucopyranosyl-(1→4)-2 acetamido-2-deoxy-β-D-glucopyranosyl-(1→4)-2 acetamido-2-deoxy-β-D-glucopyranoside 21

Compound 6 (0.02 g, 0.07 mmol) synthesized based on procedures described in Example 2 was dissolved in 1:2 DMF/THF (total volume 5 ml). To this solution, DMAP (0.011 g, 0.09 mmol), EDC (0.018 g, 0.09 mmol), and Methyl 2-amino-2-deoxy-β-D-glucopyranosyl-(1→4)-2-actemido-2-deoxy-β-D-glucopyranosyl-(1→4)-2-acetamido-2-deoxy-β-D-glucopyranosyl-(1→4)-2-acetamido-2-deoxy-β-D-glucopyranoside (0.05 g, 0.06 mmol) were added and the reaction was stirred at room temperature overnight. The reaction is represented by the following scheme:

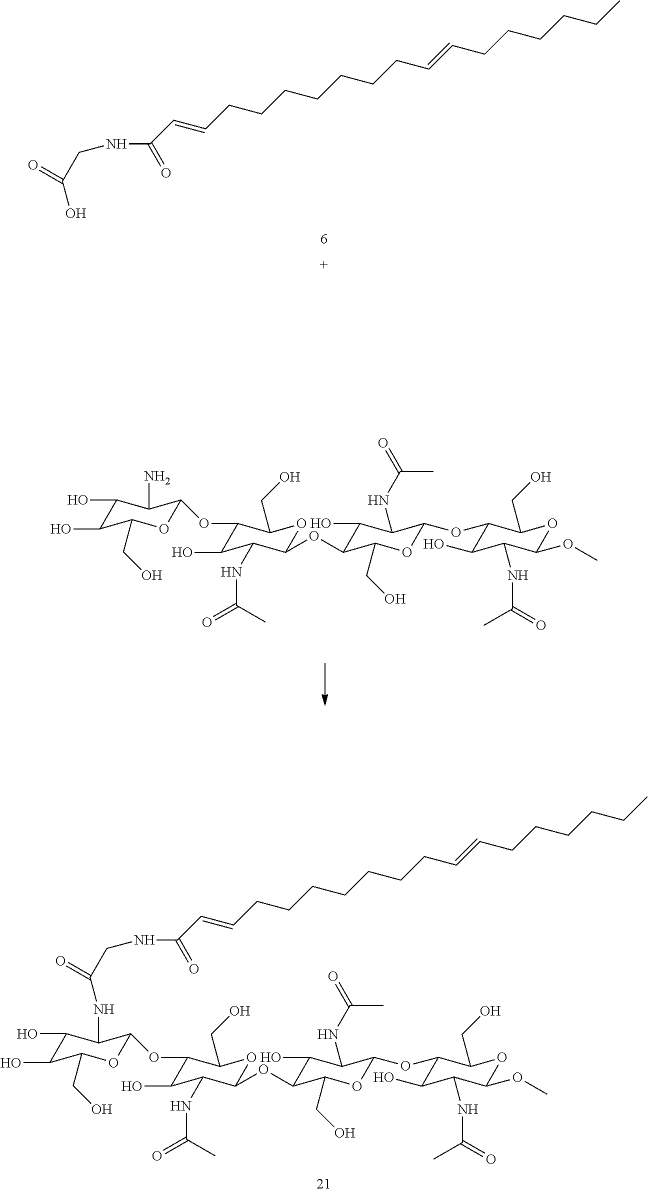 [SMILES: CCCCCC/C=C/CCCCCCC/C=C/C(=O)NCC(=O)O ; COC1OC(CO)C(OC2OC(CO)C(OC3OC(CO)C(OC4OC(CO)C(O)C(O)C4N)C(O)C3NC(C)=O)C(O)C2NC(C)=O)C(O)C1NC(C)=O ; CCCCCC/C=C/CCCCCCC/C=C/C(=O)NCC(=O)NC1C(OC2C(CO)OC(OC3C(CO)OC(OC4C(CO)OC(OC)C(NC(C)=O)C4O)C(NC(C)=O)C3O)C(NC(C)=O)C2O)OC(CO)C(O)C1O]

The latter component of the reaction mixture was synthesized based on the method described in U.S. Pat. No. 7,485,718. Then the reaction mixture was pumped dry and washed with DI-H₂O three times (1 ml/each). The final product was purified with column chromatography to provide 11 mg of desired product 21 in 16% yield. The structure was characterized by LC-MS and ¹H NMR.

¹H NMR (500 MHz, D₂O and DMSO-D⁶): δ 7.98-7.89 (m, 4H), 6.65-6.62 (m, 1H), 5.96-5.93 (m, 1H), 5.29 (br, 2H), 5.00-4.68 (m, 4H), 4.35-3.18 (m, 25H), 2.11-2.07 (m, 3H), 1.95-1.93 (m, 5H), 1.81-1.78 (m, 7H), 1.41-1.32 (m, 2H), 1.36-1.22 (m, 16H), 0.81 (br, 3H). LC-MS (ESI): m/z 1144 [M+22]⁺.

### Example 9A

### Testing of Soybean Seeds Treated with Methyl 2-deoxy-2-({[(2E,11E)-octadeca-2,11-dienoylamino]acetyl}amino)hexopyranosyl-(1→4)-2-(acetylamino)-2-deoxyhexopyranosyl-(1→4)-2-(acetylamino)-2-deoxyhexopyranosyl-(1→4)-2-(acetylamino)-2-deoxyhexopyranoside 21

Compound 21 prepared in Example 9 was evaluated using the seed germination assay described in General Material & Methods. Soybean seeds treated with this compound showed 36% germination at 20 hours with a standard deviation of 5%. Control soybean seeds showed 24% germination at 20 hours with a standard deviation of 4%.

The same compound was evaluated using the plant growth assay described Example 1. Seventy-three percent (73%) of the germinated soybean seeds treated with this compound exhibited radical lengths greater than 1.5 cm, with a standard deviation of 5%. Fifty-five percent (55%) of the control soybean seeds exhibited radical lengths greater than 1.5 cm, with a standard deviation of 9%.

Activity screen data summarized in table in case needed

| | | Germination | | | STD-Growth |
|---|---|---|---|---|---|
| Entry | Example | Yield | STD | Growth Assay | Assay |
| 1 | Control | 24% | 4% | 55% | 9% |
| 2 | Example 2 | 46% | 6% | 79% | 6% |
| 3 | Example 3 | 33% | 3% | 73% | 6% |
| 4 | Example 4 | 39% | 4% | 82% | 3% |
| 5 | Example 5 | 32% | 5% | 80% | 7% |
| 6 | Example 6 | 31% | 4% | 74% | 9% |
| 7 | Example 7 | 44% | 6% | 86% | 7% |
| 8 | Example 8 | 30% | 2% | 71% | 8% |
| 9 | Example 9 | 36% | 5% | 73% | 5% |

We claim:

1. A compound represented by the general Formula 1, 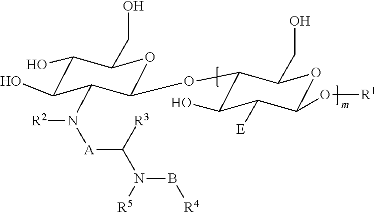
wherein the substituents are:
m is 0, 1, 2, 3 or 4;
A and B are independently selected from —C(O)—, —C(S)—, C(O)O—, —C(O)S—, —C(S)S—;
E is selected from OH, NH₂, and NHC(O)CH₃;
R¹ is selected from a linear or branched, saturated or unsaturated, hydrocarbon-based chain containing from 1 to 20 carbon atoms, arylene, or substituted arylene;
R² and R⁵ are independently selected from H and C1-20 alkyl;
R³ is selected from any side-chain of natural or unnatural amino acids, including H, C1-6 alkyl, an aryl, and a halogen;
R⁴ is selected from a linear or branched, saturated or unsaturated, heteroatom substituted or non-heteroatom substituted hydrocarbon-based chain containing from 1 to 20 carbon atoms, arylene, or substituted arylene.

2. The compound of claim 1, further defined as having the structure

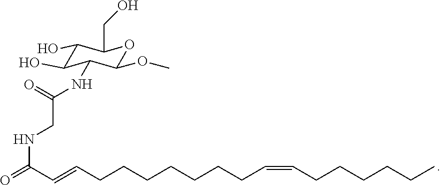 [SMILES: CCCCCC/C=C\CCCCCCC/C=C/C(=O)NCC(=O)NC1C(OC)OC(CO)C(O)C1O]

3. The compound of claim 1, further defined as having the structure

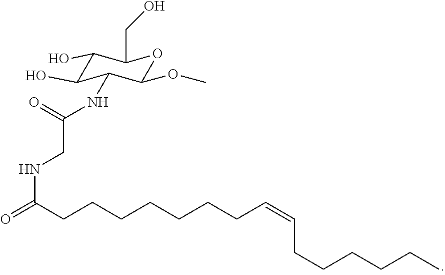 [SMILES: CCCCCC/C=C\CCCCCCCC(=O)NCC(=O)NC1C(OC)OC(CO)C(O)C1O]

4. The compound of claim 1, further defined as having the structure

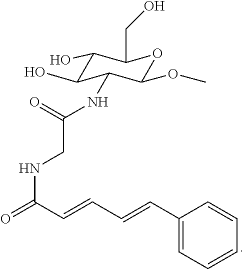 [SMILES: COC1OC(CO)C(O)C(O)C1NC(=O)CNC(=O)/C=C/C=C/c1ccccc1]

5. The compound of claim 1, further defined as having the structure

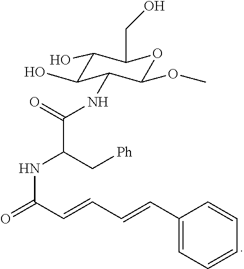 [SMILES: COC1OC(CO)C(O)C(O)C1NC(=O)C(Cc1ccccc1)NC(=O)/C=C/C=C/c1ccccc1]

6. The compound of claim 1, further defined as having the structure

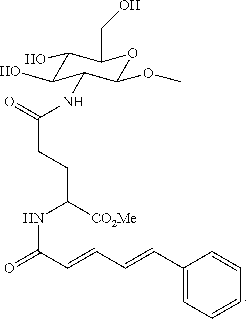 [SMILES: COC(=O)C(CCC(=O)NC1C(OC)OC(CO)C(O)C1O)NC(=O)/C=C/C=C/c1ccccc1]

7. The compound of claim 1, further defined as having the structure

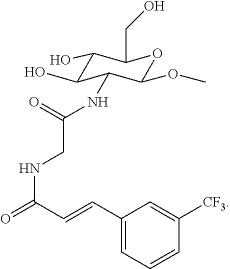 [SMILES: COC1OC(CO)C(O)C(O)C1NC(=O)CNC(=O)/C=C/c1cccc(C(F)(F)F)c1]

8. The compound of claim 1, further defined as having the structure

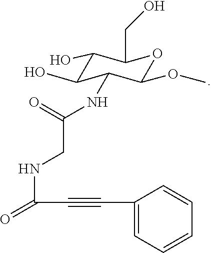 [SMILES: COC1OC(CO)C(O)C(O)C1NC(=O)CNC(=O)C#Cc1ccccc1]

9. The compound of claim 1, further defined as having the structure

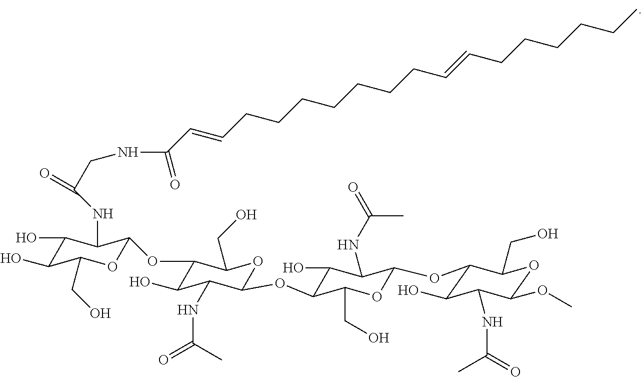 [SMILES: CCCCCC/C=C/CCCCCCC/C=C/C(=O)NCC(=O)NC1C(OC2C(CO)OC(OC3C(CO)OC(OC4C(CO)OC(OC)C(NC(C)=O)C4O)C(NC(C)=O)C3O)C(NC(C)=O)C2O)OC(CO)C(O)C1O]

10. An agricultural composition comprising the compound of claim 1, wherein the compound is present in the formulation at a concentration of 10⁻⁵ M to 10⁻¹² M.

11. The agricultural composition of claim 10, wherein the compound is present in the formulation at a concentration of about 10⁻⁷ M.

12. An agricultural composition comprising the compound of claim 1, wherein the composition is applied to propagating material of a plant.

13. The composition of claim 12, wherein the plant is a legume.

14. The composition of claim 13, wherein the legume is soybean.

15. The composition of claim 12, wherein the composition is applied to propagating material of the plant to provide improved growth.

16. The composition of claim 12, wherein the propagating material is seed.

17. The composition of claim 15, wherein the composition is applied to seed to accelerate the rate of germination.

18. The composition of claim 12, further comprising one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones applied to the propagating material.

19. An agricultural composition comprising the compound of claim 1, wherein the composition is applied to foliage.

20. The composition of claim 19, further comprising one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones applied to the foliage.

21. A method for treating a plant, comprising applying a composition represented by the general Formula 1, 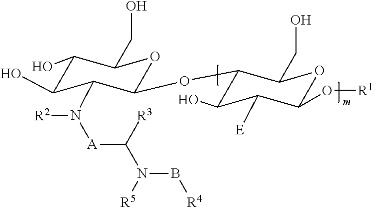
wherein the substituents are:
m is 0, 1, 2, 3 or 4;
A and B are independently selected from —C(O)—, —C(S)—, C(O)O—, —C(O)S—, —C(S)S—;
E is selected from OH, NH₂, and NHC(O)CH₃;
R¹ is selected from a linear or branched, saturated or unsaturated, hydrocarbon-based chain containing from 1 to 20 carbon atoms, arylene, or substituted arylene;
R² and R⁵ are independently selected from H and C1-20 alkyl;
R³ is selected from any side-chain of natural or unnatural amino acids, including H, C1-6 alkyl, an aryl, and a halogen;
R⁴ is selected from a linear or branched, saturated or unsaturated, heteroatom substituted or non-heteroatom substituted hydrocarbon-based chain containing from 1 to 20 carbon atoms, arylene, or substituted arylene.

22. The method for treating a plant of claim 21, wherein the composition is further defined as having the structure

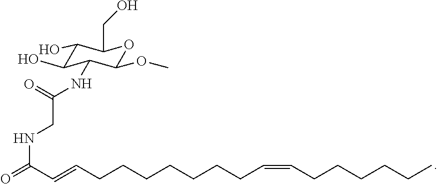 [SMILES: CCCCCC/C=C\CCCCCCC/C=C/C(=O)NCC(=O)NC1C(OC)OC(CO)C(O)C1O]

23. The method for treating a plant of claim 21, wherein the composition is further defined as having the structure

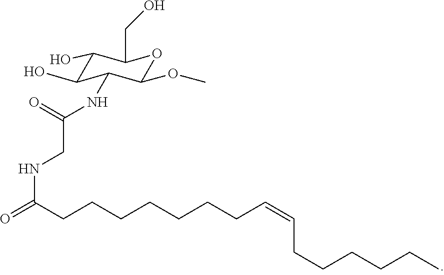 [SMILES: CCCCCC/C=C\CCCCCCCC(=O)NCC(=O)NC1C(OC)OC(CO)C(O)C1O]

24. The method for treating a plant of claim 21, wherein the composition is further defined as having the structure

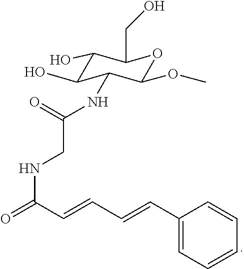 [SMILES: COC1OC(CO)C(O)C(O)C1NC(=O)CNC(=O)/C=C/C=C/c1ccccc1]

25. The method for treating a plant of claim 21, wherein the composition is further defined as having the structure

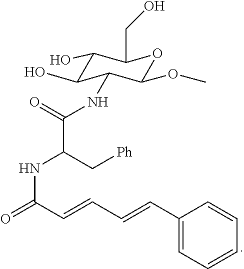 [SMILES: COC1OC(CO)C(O)C(O)C1NC(=O)C(Cc1ccccc1)NC(=O)/C=C/C=C/c1ccccc1]

26. The method for treating a plant of claim 21, wherein the composition is further defined as having the structure

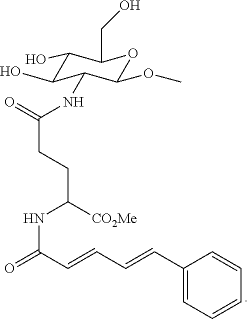 [SMILES: COC(=O)C(CCC(=O)NC1C(OC)OC(CO)C(O)C1O)NC(=O)/C=C/C=C/c1ccccc1]

27. The method for treating a plant of claim 21, wherein the composition is further defined as having the structure

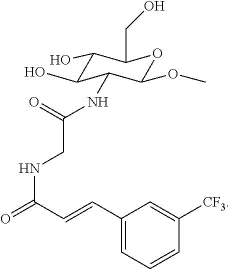 [SMILES: COC1OC(CO)C(O)C(O)C1NC(=O)CNC(=O)/C=C/c1cccc(C(F)(F)F)c1]

28. The method for treating a plant of claim 21, wherein the composition is further defined as having the structure

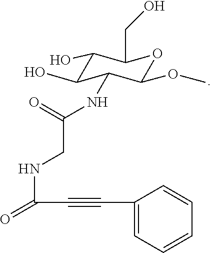 [SMILES: COC1OC(CO)C(O)C(O)C1NC(=O)CNC(=O)C#Cc1ccccc1]

29. The method for treating a plant of claim 21, wherein the composition is further defined as having the structure

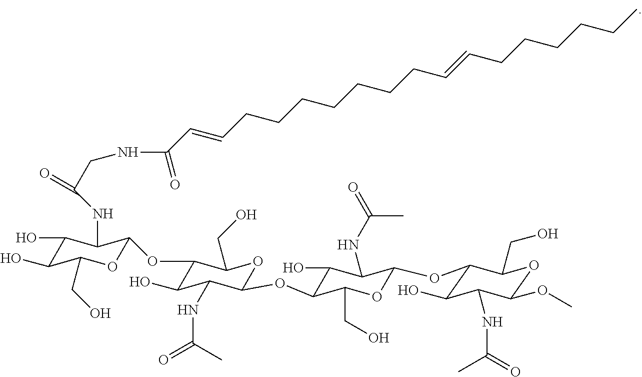 [SMILES: CCCCCC/C=C/CCCCCCC/C=C/C(=O)NCC(=O)NC1C(OC2C(CO)OC(OC3C(CO)OC(OC4C(CO)OC(OC)C(NC(C)=O)C4O)C(NC(C)=O)C3O)C(NC(C)=O)C2O)OC(CO)C(O)C1O]

30. The method of claim 21, wherein the composition is applied as a seed coating.

31. The method of claim 21, wherein the composition is applied to foliage.

32. The method of claim 21, wherein the composition is applied to soil either prior to or following planting plant propagating material.

33. The method of claim 21, wherein the composition is applied to a dicot.

34. The method of claim 33, wherein the composition is applied to soybean.

35. The method according to claim 21, further comprising one or more insecticides, fungicides, nematocides, bactericides, acaricides, herbicides, plant nutrients, growth regulators such as rooting stimulants, chemosterilants, semiochemicals, repellents, attractants, pheromones, feeding stimulants, other biologically active compounds, microbial inocula or entomopathogenic bacteria, viruses or fungi applied to the plant.

36. An agricultural composition comprising the compound of any of claims 1 to 9, wherein the compound is present in the formulation at a concentration of 10⁻⁵ M to 10⁻¹² M.

37. The agricultural composition of claim 36, wherein the compound is present in the formulation at a concentration of about 10⁻⁷ M.

38. An agricultural composition comprising the compound of any of claims 1 to 9, wherein the composition is applied to propagating material of a plant.

39. The composition of claim 38, wherein the plant is a legume.

40. The composition of claim 39, wherein the legume is soybean.

41. The composition of claim 38, wherein the composition is applied to propagating material of the plant to provide improved growth.

42. The composition of claim 38, wherein the propagating material is seed.

43. The composition of claim 42, wherein the composition is applied to seed to accelerate the rate of germination.

44. The composition of claim 38, further comprising one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones applied to the propagating material.

45. An agricultural composition comprising the compound of claim 1, wherein the composition is applied to foliage.

46. The composition of claim 45, further comprising one or more insecticides, fungicides, nematocides, bactericides, acaricides, entomopathogenic bacteria, viruses or fungi, growth regulators such as rooting stimulants, chemosterilants, repellents, attractants, pheromones, feeding stimulant and other signal compounds including, but not limited to, apocarotenoids, flavonoids, jasmonates and strigolactones applied to the foliage.
